(19) United States
(12) Patent Application Publication
(10) Pub. No.: US 2019/0241913 A1
(43) Pub. Date: Aug. 8, 2019

(54) METHOD FOR PRODUCTION OF ISOPRENE RECOMBINANT MICROORGANISM, GENE CONSTRUCT, VECTOR AND APPLICATION THEREOF

(71) Applicant: Reliance Industries Limited, Mumbai (IN)

(72) Inventors: Manmeet Ahuja, Punjab (IN); Mansi Pankaj Vora, Mumbai (IN); Jasmine Isar, Faridabad (IN); Harshvardhan Joshi, Gwalior (IN); Sneh Sanjay Badle, Thane (IN); Jayesh Suman Varavadekar, Thane (IN); Dharmendra Ambalal Jain, Thane (IN); Piyush Prafull Sethia, Mumbai (IN); Hari Krishna Reddy Chigullarevu, Anantapur (district) (IN); Rachana Suresh Rathod, Mumbai (IN); Shrikant Balkisan Dhoot, Navi Mumbai (IN); Vidhya Rangaswamy, Thane (IN)

(21) Appl. No.: 16/340,525

(22) PCT Filed: Oct. 12, 2017
(86) PCT No.: PCT/IB2017/056316

§ 371 Date: Apr. 9, 2019

(30) Foreign Application Priority Data
Oct. 12, 2016 (IN) 201621027569

(51) Int. Cl. C12P 5/00; C07K 14/245; C12N 9/02; C12N 15/52; C12N 9/10; C12N 9/12; C12N 9/88; C12N 9/90

(52) U.S. Cl. CPC C12P 5/007; C07K 14/245; C12N 9/0093; C12N 15/52; C12N 9/1022; C12N 9/1241; C12Y 503/03002; C12N 9/90; C12Y 117/07001; C12Y 202/01007; C12Y 207/0704; C12Y 402/03027; C12Y 406/01012; C12N 9/88

(57) ABSTRACT

The present disclosure relates to method for enhanced production of metabolite including but not limiting to isoprene and isoprenoid through chromosomal integration of genes belonging to MEP pathway. The disclosure further relates a host cell for the production of the said metabolite. The method of the present disclosure bypasses the cumbersome method of plasmid application for the production of metabolite. The disclosure also relates to a gene construct comprising MEP genes and auxotrophic markers and a vector comprising the said gene construct.

### TECHNICAL FIELD

The present disclosure broadly relates to a method for metabolite production. The disclosure particularly relates to recombinant methods for the production of metabolite including but not limiting to isoprene and isoprenoid. The disclosure more particularly relates to chromosomal integration of genes belonging to 2-C-methyl-D-erythritol 4-phosphate (MEP) pathway for production of metabolite including but not limiting to isoprene and isoprenoid.

The disclosure also relates to a recombinant microorganism (host cell) comprising component selected from a group comprising chromosome of the said microorganism integrated with genes belonging to MEP pathway, superoperon and Phage Lambda derived RED Recombination system, or any combination thereof, for the production of the metabolite.

The disclosure further relates to a gene construct comprising genes belonging to MEP pathway, and auxotrophic markers. The disclosure also relates to a vector comprising the said gene construct for the production of the metabolite.

### BACKGROUND OF THE DISCLOSURE

Isoprene is naturally produced volatile cell metabolite emitted from the leaves of any plant species. The commercially valuable isoprenoid family of metabolites is formed from isoprene which serves as a monomeric building block. Isoprenoids are seldom synthesized chemically except for isoprene. Being gaseous above 34° C., harvesting isoprene from plants is not feasible and therefore is exclusively made through chemical synthesis from petrochemicals.

Also, there are biological processes identified for the production of isoprene such as synthetic biology and metabolic engineering. In the said biological processes, the metabolic burden from DNA, RNA and protein synthesis of the cell increases if it has to maintain multiple plasmids. And, the burden is further increased due to the total number of antibiotic resistance proteins that the cell has to produce, leading to low yields of the desired metabolite. Further, plasmid based expression systems have several drawbacks such as segregational in-stability or allele segregation and possible structural instability which reduces the amount of production of product of interest. Additionally, antibiotics required for selecting and maintaining plasmids in the host during fermentation result in increased costs. The instant specification described herein intends to overcome the limitations observed in the general art.

### STATEMENT OF THE DISCLOSURE

The present disclosure relates to a method for production of metabolite including but not limiting to isoprene and isoprenoid by chromosomal integration of genes belonging to MEP pathway.

In another embodiment, disclosure relates to a method for production of metabolite including but not limiting to isoprene and isoprenoid by combining chromosome integrated with a gene belonging to MEP pathway and at least one component selected from a group comprising superoperon, phage Lambda derived RED recombination system.

In a particular embodiment, the present disclosure relates to a method for enhancing production of the metabolite including but not limiting to isoprene and isoprenoid by chromosomal integration of copies of gene belonging to MEP pathway by using auxotrophic markers and fusion PCR.

The disclosure also provides insight to the effect of copy numbers of genes belonging to MEP pathway integrated to the chromosome for enhanced production of metabolite including but not limiting to isoprene and isoprenoid.

### BRIEF DESCRIPTION OF ACCOMPANYING FIGURES

FIG. 1 illustrates the scheme of the MEP and DXP pathways

FIG. 2 illustrates genome map with locations of MEP pathway genes distributed across the genome of A) E. coli and B) B. subtilis.

FIG. 3 illustrates creation of ‘5′lacZ-P_{tac} ispS-camR-3′lacZ’ fusion fragment by fusion PCR.

FIG. 4 illustrates one copy each of ispS, fni and dxs introduced successively at different loci in the genome of E. coli.

FIG. 5 illustrates gene construct of MEP genes and auxotrophs for chromosome integration.

FIG. 6 illustrates stepwise construction of superoperon.

FIG. 7 illustrates plasmids, wherein A) illustrates pET30a ispS with Tag; B) illustrates pET30a ispS without Tag; C) illustrates pET30a dxs with Tag; D) illustrates pET28a-dxs without Tag; E) illustrates pET30a fni with Tag; and F) illustrates pET28a fni without Tag; and G) illustrates pET30a dxr with His Tag.

FIG. 8 illustrates stepwise cloning by Fusion PCR for integration of multiple genes in a single plasmid.

FIG. 9 illustrates multiple gene constructs for isoprene biosynthesis in plasmid pET28a, wherein A) illustrates pET28a-T7-dxs-RBS-ispG(E. coli)-T7-ispS-RBS-fni; B) illustrates pET28a-T7-dxs-RBS-ispG-T7-ispS-RBS-fni; C) illustrates pET28a-T7-dxs-RBS-ispG (EC)-T7-fni-RBS-ispS; D) illustrates pET28a-T7-dxs-RBS-ispG-T7-fni-RBS-ispS E) illustrates pET28a-T7-dxs-T7-ispS-RBS-fni and F) illustrates pET28a-T7-fni-T7-ispS

FIG. 10 illustrates protein expression of pET28a (+)-T7-dxs-RBS-ispG (B. subtilis)-T7-ispS-fni in E. coli BL21(DE3), wherein Lane 1 is Marker; Lanes 2 to 4 are E. coli BL21-DE3, at 0 hour, 1 hour and 2 hours post induction; Lanes 5 to 7 are E. coli BL21-DE3-pET28a (+)-T7-dxs-RBS-ispG-T7-ispS-fni Colony-1 at 0 hour, 1 hour and 2 hours post induction; and Lanes 8 to 10 are E. coli BL21-DE3-pET28a (+)-T7-dxs-RBS-ispG-T7-ispS-fni Colony-2 at 0 hour, 1 hour and 2 hours post induction.

FIG. 11 illustrates protein expression pET28a (+)-T7-dxs-RBS-ispG(E. coli)-T7-ispS-fni in E. coli BL21(DE3), wherein Lane 1 is Marker; Lane 2 is E. coli BL21 (DE3); Lane 3 is E. coli BL21 (DE3)-pET28a-dxs (without Tag); Lane 4 is E. coli BL21 (DE3)-pET28a-fni (without Tag); Lane 5 is E. coli BL21 (DE3)-pET30a-ispS(without Tag); and Lanes 6 and 7 are E. coli BL21 (DE3)-pET28a-T7-dxs-RBS-ispG(E. coli)-T7-ispS-RBS-fni of colony-1 and colony-2, respectively.

FIG. 12 illustrates detection of FNI protein expression in pET28a (+)-T7-dxs-RBS-ispG-T7-ispS-fni, wherein lane 1 is BL21 (DE3)-pET28a-fni (500 ng); Lanes 2 to 5 are E. coli BL21 (DE3)—pET28a (+)-T7-dxs-RBS-ispG (B. subtilis)-T7-ispS-fni of colony-1 at 0 hour, 1 hour, 2 and 3 hours post induction; Lanes 6 to 9 are E. coli BL21 (DE3)—pET28a (+)-T7-dxs-RBS-ispG (B. subtilis)-T7-ispS-fni of colony-2 at 0 hour, 1 hour, 2 hours and 3 hours post induction; and Lane 10 is Marker.

FIG. 13 illustrates detection of FNI protein expression in pET28a (+)-T7-dxs-RBS-ispG(E. coli)-T7-ispS-fni in E. coli BL21(DE3), wherein Lane 1 is BL21 (DE3)-pET28a-fni (500 ng); Lanes 2 to 5 are E. coli BL21 (DE3)—pET28a (+)-T7-dxs-RBS-ispG (E. coli)-T7-ispS-fni of colony-1 at 0 hour, 1 hour, 2 hours and 3 hours post induction; Lanes 6 to 9 are E. coli BL21 (DE3)—pET28a (+)-T7-dxs-RBS-ispG (E. coli)-T7-ispS-fni of colony-2 at 0 hour, 1 hour, 2 hour and 3 hours post induction; and Lane 10 is Marker.

FIG. 14 illustrates protein expression of i) pET28a (+)-T7-dxs-RBS-ispG(B. subtilis)-T7-fni-RBS-ispS, ii) pET28a (+)-T7-dxs-RBS-ispG(E. coli)-T7-fni-RBS-ispS, iii) pET28a (+)-T7-dxs-T7-fni-RBS-ispS, and iv) pET28a (+)-T7-dxs-T7-ispS-RBS-fni in E. coli BL21(DE3), wherein Lane 1 is Marker; Lane 2 is E. coli BL21 (DE3); Lane 3 is E. coli BL21 (DE3)-pET28a-dxs (without Tag); Lane 4 is E. coli BL21 (DE3)-pET28a-fni (without Tag); Lane 5 is E. coli BL21 (DE3)-pET30a-ispS (without Tag); Lane 6 is E. coli BL21 (DE3)-pET28a-T7-dxs-RBS-ispG (B. subtilis)-T7-ispS-RBS-fni, Lane 7 is E. coli BL21 (DE3)-pET28a-T7-dxs-RBS-ispG (B. subtilis)-T7-fni-RBS-ispS, Lane 8 is E. coli BL21 (DE3)-pET28a-T7-dxs-RBS-ispG (E. coli)-T7-fni-RBS-ispS, Lane 9 is E. coli BL21 (DE3)-pET28a-T7-dxs-T7-fni-RBS-ispS, and Lane 10 is E. coli BL21 (DE3)-pET28a-T7-dxs-T7-ispS-RBS-fni, and wherein A) illustrates protein expression in 10% polyacrylamide gel and B) illustrates protein expression in 12% polyacrylamide gel.

FIG. 15 illustrates fermentation profile of the major and minor products of modified E. coli K-12 strain

FIG. 16 illustrates intracellular metabolites, OD at 600 and isoprene production profile of modified E. coli K-12 strain.

FIG. 17 illustrates fermentation profile of the major and minor products of modified E. coli K12 strain with intermittent IPTG induction.

FIG. 18 illustrates fermentation profile of the major and minor products of modified E. coli MG1655 strain at about 40° C.

FIG. 19 illustrates fermentation profile of the major and minor products of modified E. coli BL21 strain

FIG. 20 illustrates intracellular metabolites, OD600 and isoprene production profile of modified E. coli BL21 strain.

FIG. 21 illustrates fermentation profile of the major and minor products of modified E. coli BL21 strain (without induction).

FIG. 22 illustrates fermentation profile of the major and minor products of modified E. coli BL21 strain using terrific broth with glycerol as feed.

FIG. 23 illustrates the major and minor products during isoprene production during Fed Batch fermentation for isoprene production by MG1655 strain with three copies of ispS, and two copies dxs and fni genes

FIG. 24 illustrates the intracellular metabolites produced during isoprene production during Fed Batch fermentation for isoprene production by MG1655 strain with three copies of ispS, and two copies dxs and fni genes

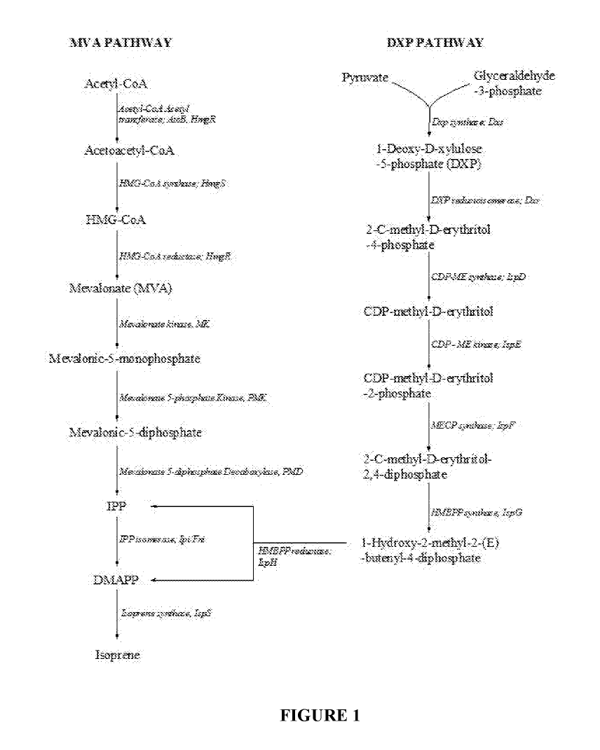

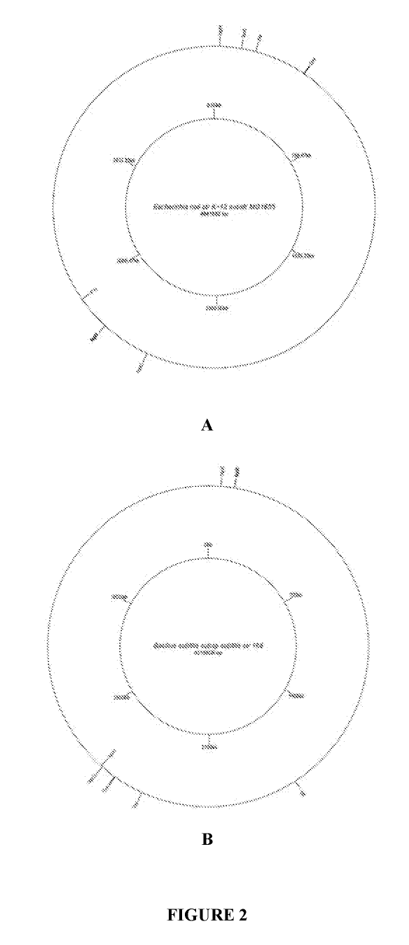

### DETAILED DESCRIPTION OF THE DISCLOSURE

The present disclosure relates to a method for production of metabolite including but not limiting to isoprene and isoprenoid in host cell such as microorganism including but not limiting to bacteria and fungus.

The present disclosure relates to a method for production of metabolite including but not limiting to isoprene and isoprenoid by microorganism including but not limiting to bacteria and fungus by over expression of genes belong to MEP pathway optionally along with recombineering system including but not limiting to Lambda-RED recombinase system or superoperon, or any combination thereof.

The genes belonging to MEP pathway used in the method for expression of isoprene and isoprenoid includes but is not limiting to endogenous genes of MEP pathway and heterologous genes of MEP pathway.

In an exemplary embodiment, the present disclosure relates to a method for production of metabolite including but not limiting to isoprene and isoprenoid in a microorganism including but not limiting to bacteria and fungus by over expression of at least one of endogenous gene of MEP pathway or heterologous genes of MEP pathway, or a combination thereof.

In a particular embodiment, the present disclosure relates to a method for enhanced production of metabolite including but not limiting to isoprene and isoprenoid in a microorganism including but not limiting to bacteria and fungus by over expression of at least one of endogenous gene of MEP pathway or heterologous genes of MEP pathway.

In an embodiment, the present disclosure particularly relates to a method for production of the metabolite including but not limiting to isoprene and isoprenoid by integration of genes belonging to MEP pathway constructed by fusion PCR into the chromosome of the microorganism including but not limiting to bacteria and fungus by Lambda RED mediated homologous recombination.

In a particular embodiment, the microorganism comprising the chromosome integrated with genes belonging to MEP pathway optionally comprises recombineering system including but not limiting to Lambda-RED recombinase system and superoperon, for the enhanced production of the metabolite.

In an embodiment, the microorganism comprising the chromosome integrated with genes belonging to MEP pathway comprises recombineering system including but not limiting to Lambda-Red recombinase system and superoperon, for the enhanced production of the metabolite.

In a non-limiting embodiment, the fusion PCR employed in the method of the instant disclosure provides the option of making large constructs with different DNA fragments without requiring any cloning system. And, since, linear fragments are required for Lambda RED recombineering, the fusion PCR reaction reduces the time period required for making each construct, which is used in the enhancement of production of the metabolite.

In an embodiment, the genes belonging to MEP pathway for integration in to the chromosome of the microorganism is selected from a group comprising dvs, fni/idi, dxr, ispG, ispS, ispD, ispH and ispF, or any combination thereof.

In an embodiment, the Lambda-Red recombinase system is derived from the bacteriophage Lambda consisting of three genes exo, ft, and gam. The RED genes along with the P_{tac} promoter are amplified from the pKM208 plasmid. The upstream and downstream regions of the locus amyA are amplified from E. coli gDNA. The antibiotic selection marker is amplified from plasmid pGEX2T. A fusion fragment consisting of the RED genes, an ampicillin antibiotic resistance marker and the upstream and downstream regions of the amyA gene is constructed. It is a powerful tool for making targeted genetic changes such as gene deletions and insertions with ease.

The present disclosure, relates to a method for enhanced production of metabolite selected from a group comprising isoprene and isoprenoid, or a combination thereof, said method comprising steps of:
transforming host cell with gene construct comprising 2-C-methyl-D-erythritol 4-phosphate (MEP) genes; and integrating the MEP genes into chromosome of the host cell, thereby enhancing production of the metabolite selected from the group comprising isoprene and isoprenoid.

In an embodiment, the host cell is selected from a group comprising bacteria and fungi.

In an embodiment, the host cell is selected from a group comprising E. coli K-12, E. coli K-12 MG1655 and E. coli BL21.

In an embodiment, the MEP genes is selected from a group comprising dxs, fni, idi, dxr, ispG, ispS, ispD, and ispF, or any combination thereof.

In an embodiment, the MEP genes are fused by fusion PCR prior to transformation into the host cell, wherein the MEP genes are fused in a combination selected from a group comprising—
isps and fni;
isps, fni and dxs;
isps, fni, dxs, dxr and
isps, fni, dxs, ispD;

In an embodiment, the fused MEP genes comprises auxotrophic markers selected from a group comprising thyA, metB, glnA, trpA, leuA, tyrA, lysA and proC, or a combination thereof.

In an embodiment, the host cell comprises lambda RED recombination system and superoperon.

In an embodiment, the superoperon comprises MEP genes separated by internal ribosome entry site and accessory genes selected from a group comprising Fe—S cluster interacting redox polypeptides and co-factor balancing genes, or a combination thereof.

In an embodiment, the integration of the MEP genes with the chromosome of the host cell is by lambda RED recombination system in the host cell.

In an embodiment, the volumetric productivity of the isoprene is ranging from about 86.0 mg L⁻¹ h⁻¹ to 102 mg L⁻¹ h⁻¹ and the specific productivity of the isoprene is ranging from about 3.26 mg g⁻¹ h⁻¹ to 8.27 mg g⁻¹ h⁻¹.

The disclosure further relates to a gene construct comprising MEP genes and auxotrophic marker or antibiotic resistance marker or a combination thereof.

In an embodiment, the MEP genes in the gene construct are selected from a group comprising dxs, fni, idi, dxr, ispG, ispS, ispD, and ispF, or any combination thereof; and the auxotrophic marker is selected from a group comprising Fe—S cluster interacting redox polypeptides and co-factor balancing genes, or a combination thereof.

In an embodiment, the gene construct comprises MEP genes in a combination selected from a group comprising—
isps and fni;
isps, fni and dxs;
isps, fni, dxs, dxr and
isps, fni, dxs, ispD;
along with the auxotrophic marker

The disclosure further relates to a host cell comprising chromosome integrated with MEP genes, lambda RED recombination system and superoperon.

In an embodiment, the host cell is selected from a group comprising bacteria and fungi.

In an embodiment, the host cell is selected from a group comprising E. coli K-12, E. coli K-12 MG1655 and E. coli BL21.

In an embodiment, the superoperon comprises MEP genes separated by internal ribosome entry site and accessory genes selected from a group comprising Fe—S cluster interacting redox polypeptides and co-factor balancing genes, or a combination thereof.

In an embodiment, the cell produces enhanced isoprene with volumetric productivity of the isoprene ranging from about 86.1 mg L⁻¹ h⁻¹ to 102.1 mg and the specific productivity of the isoprene ranging from about 3.26 mg g⁻¹ h⁻¹ to 8.27 mg g⁻¹ h⁻¹.

The disclosure further relates to a vector comprising the gene construct having MEP genes in the combination as defined above.

In an embodiment, the superoperon approach is used to down regulate the expression of genes belonging to terpenoid biosynthesis pathway including but not limiting to ispA, ispB and ispU. The superoperon stitches multiple genes with their RBSs either under a single promoter or individual promoters.

In another embodiment, the superoperon comprises genes belonging to MEP pathway, wherein the genes of MEP pathway are selected from a group comprising dxs, fni/idi, dxr, ispG, ispS, ispD, and ispF, or any combination thereof. The said genes of the MEP pathway in the superoperon are under the influence of a promoter selected from a group comprising P_{tac}. The genes of the MEP pathway in the superoperon are separated by IRES (internal ribosome entry site) or RBS (Ribosome binding site) with suitable spacers for optimal expression causing increase in the production of the metabolite including but limiting to isoprene and isoprenoid. The RBS employed is either the strong B. subtilis gsiB RBS or designed RBS calculator v2.0.

In an alternate embodiment superoperon comprises accessory genes selected from a group comprising regulators, Fe—S cluster interacting redox polypeptides and co-factor balancing genes, or a combination thereof, wherein the accessory genes optimizes flux in the microorganism, particularly carbon flux. In the present invention, the isoprene production is further improved by modulating the accessory genes of the superoperon.

In an embodiment, the genes belonging to MEP pathway are integrated at multiple loci of the chromosome of the microorganism including but not limiting to bacteria and fungi by using auxotrophy selection marker, wherein the auxotrophy markers of the instant invention for integration of the genes belonging to MEP pathway bypasses the use of antibiotic selection marker.

The use of auxotrophy markers for integration of the genes belonging to MEP pathway into the chromosome of the microorganism, bypasses the use of antibiotic selection makers.

In an embodiment, the auxotrophy markers for integration of the genes belonging to MEP pathway are selected from a group of genes involved in amino acid metabolism comprising but not limiting to thyA, metB, glnA, trpA, leuA, tyrA, lysA and proC, or genes from any other amino acid metabolism or combination thereof. Apart from the amino acid genes, knockouts of sugar utilization genes selected from a group comprising xylA, araA or any other sugar utilization genes can also be used to create auxotroph's for chromosomal integration the genes.

In an embodiment, the auxotrophy markers for integration of the genes belonging to MEP pathway increases copy numbers of the genes in the chromosome of the microorganism. Use of auxotrophy markers for the said chromosome integration bypasses the requirement of using antibiotics as selection markers.

In another embodiment, the fusion PCR employed in the instant invention creates one fragment with all the genes belonging to MEP pathway to enable the integration at the chromosomal locus.

In an alternate embodiment, the fusion PCR employed in the instant invention creates at least two fragments comprising genes belonging to MEP pathway with overlapping regions, to enable the integration at the chromosomal locus.

In another alternate embodiment, the fusion PCR employed in the instant invention creates multiple fragments comprising genes belonging to MEP pathway with overlapping regions, to enable the integration at the chromosomal locus.

In an embodiment, the chromosomal integration of genes belonging to MEP pathway for the enhancement in the production of the metabolite including isoprene and isoprenoid in the host bypasses the plasmid approach for expression of the metabolite, thereby reduces the vector burden in the microorganism.

In an embodiment, the method for enhancement in the production of metabolite including but not limiting to isoprene and isoprenoids by chromosomal integration of genes belonging to MEP pathway, optionally along with recombineering system including but not limiting to Lambda-RED recombinase system, or superoperon, or any combination thereof is dependent on the presence of carbon flux such as glucose or related sugar molecule.

In another embodiment, the genes belonging to MEP pathway integrated into the chromosome of the microorganism are codon-optimized to further enhance the production of the metabolite including but not limiting to isoprene and isoprenoid.

In a non-limiting embodiment, MEP pathway genes, such as dxs, fni/idi, dxr, ispG, ispS, ispD, and ispF are codon optimized to further enhance the production of metabolite including but not limiting to isoprene and isoprenoid.

In an embodiment, the microorganism is selected from a group comprising E. coli K-12, E. coli K-12 MG1655 and E. coli BL21 (DE3), wherein the chromosome of the said microorganism is integrated with genes belonging to MEP pathway, such as dxs, fni/idi, dxr, ispG, ispS, ispD, and ispF and the said microorganism optionally comprises recombineering system including but not limiting to Lambda-RED recombinase system, or superoperon, or a combination thereof.

In another embodiment, the microorganism selected from a group comprising E. coli K-12 E. coli K-12 MG1655 and E. coli BL21 (DE3) is transformed with pTP223, plasmid carrying λ-RED with tetracycline resistance marker. The transformed microorganism is grown and k-RED gene is induced with 1 mM IPTG followed by heat shock at temperature of about 42° C. The transformed cells are made electrocompetent and electroporated with the k-RED. The insertion of the k-RED at the desired locus is screened by testing the microorganisms with antibiotic resistance or auxotrophy and further confirmed using colony polymerase chain reaction (PCR).

In another embodiment, microorganism selected from a group comprising E. coli K-12, E. coli K-12 MG1655 and E. coli BL21 (DE3) comprises T7-RNA polymerase.

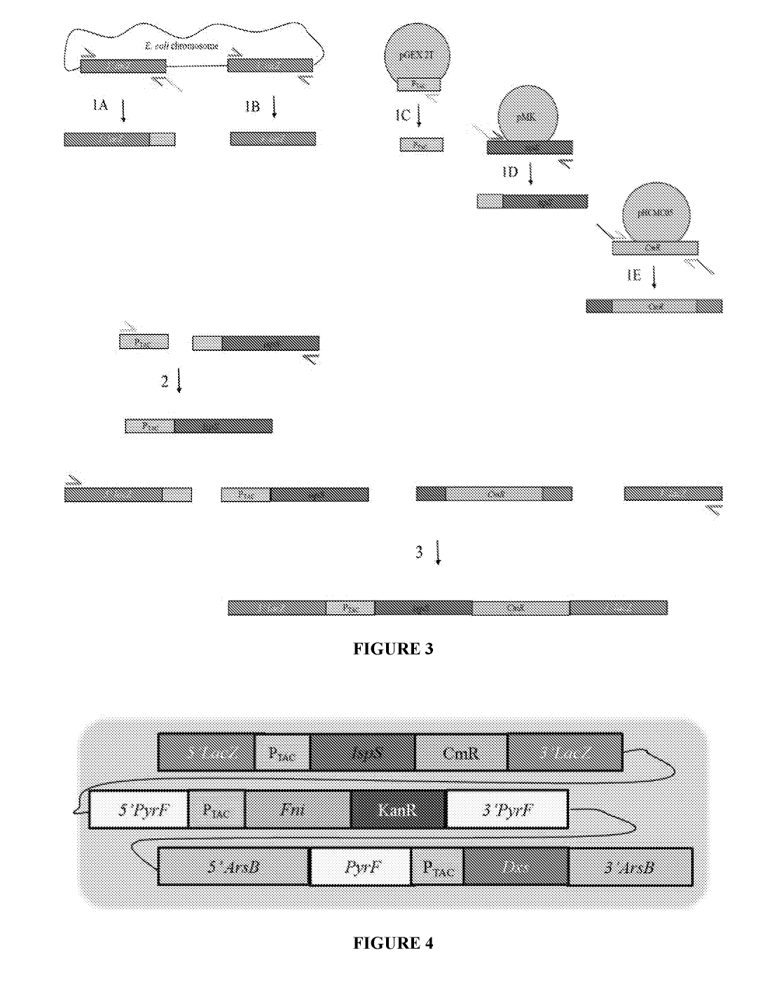

In an embodiment, the chromosomal integration of genes belonging to MEP pathway for the production of metabolite including but not limiting to isoprene and isoprenoid is carried out through fusion polymerase chain reaction (PCR). The fusion PCR enables the fusion of up to six different genes, preferably six different genes belonging to MEP pathway or more genes, by bypassing the cumbersome cloning procedures and shortening refractory periods between the amplification of fragments and transformation. FIG. 3 illustrates creation of a fusion fragment for the ispS gene with flanking regions and antibiotic marker. FIG. 8 illustrates the stepwise cloning procedure using fusion PCR for multiple genes in a single plasmid

In another embodiment, the chromosomal integration of genes belonging to MEP pathway for the production of metabolite including but not limiting isoprene and isoprenoid is carried out through fusion PCR. The fusion PCR enables the fusion of at least 2 genes belonging to MEP pathway by passing the cumbersome cloning procedures and shortening refractory periods between the amplification of fragments and transformation.

In an embodiment, the ratio of integration of genes belonging to MEP pathway for the production of the metabolite on the chromosomal loci of the microorganism is at least one copy each of ispS, fni and dxs and any combination of ispDF, ispG or ispH.

The ispG and ispH code for iron-sulfur cluster proteins and its activity is dependent on the genes of the microorganism such as fidA and fpr encoding flavodoxin and flavodoxin reductase which is responsible for electron transfer.

The chromosomal integration of genes belonging to MEP pathway optionally along with recombineering system including but not limiting to Lambda-RED recombinase system and superoperon, in the methods of the present disclosure enhances the production of metabolite including but not limiting to isoprene and isoprenoid. However, efficient production of the metabolite is not only dependent on the number of copies of the MEP pathway genes but also on many other factors such as precursor and co-factor availability and loss of carbon to secondary metabolites. However, the instant invention provides certain strategies to optimize the said limitation to maintain the enhancement of the metabolite by the chromosomal integration, the strategies are—
Precursor balancing, which increases the flux through pathway that result in increased precursor availability for MEP pathway.
Reducing carbon loss by deleting of genes/pathways responsible for production of secondary metabolites such as acetate, lactate and formate which is wasteful without affecting the overall growth of the microorganism.
Increasing energy equivalent by increasing the availability of NADPH and ATP pool of the cells by manipulating pathways and genes responsible for their generation enabling more precursor to be processed via the MEP pathway
Optimizing growth rate by over-expression and knockouts of genes that bring about a change in carbon utilization and growth rate which is optimal for the metabolite, and
Manipulation of accessory genes and regulators associated with genes belonging to MEP pathway or, by overexpression and knockouts of said accessory genes and regulators. Table 1 lists the gene targets for knockouts and table 2 lists the gene targets for overexpression.

TABLE 1
Gene targets for knockouts

| Sr. | | Process/Molecule |
|---|---|---|
| No | Gene Name | targeted |
| 1 | iclr; arcA | Metabolism |
| 2 | cra | Sugars |
| 3 | atpFH; adhE; sucA; poxB; ldhA; | Pyruvate |
| | frdBC; pflB; ackA | |
| 4 | ctfB; pta; buk; | Butanol |
| 5 | ldhA; pflB | Secondary metabolite |
| | | formation |
| 6 | pts1 | Metabolism |
| 7 | pgi; zwf; gnd; pckA; ppc; lpdA | Metabolism |
| | pyk | |
| 8 | cyaA; pts1; crr; pfkA; pgi; ptsG; IhfA; | Metabolism |
| | IhfB; Fis; pstH; atpCDEF; sucA; | |
| | sucB; lpdA; sdhCDAB | |
| 9 | maeB; frdA; pta; poxB; ldhA; zwf; | Ethanol |
| | ndh; mdh; sfcA | |
| 10 | galK | Isoprene |
| 11 | tdh; tdC; sst | Threonine |
| 12 | deoB; yhfW; yahI; pta; eutD; arcC; | Lycopene |
| | yqeA; gdhA; ppc; pta; serA; thrC | |
| 13 | sr1; gapB; pckA; gapA; ccpn | Metabolism |
| 14 | ldhA; pflB; ptsG; | Succinate |
| 15 | ppsA; poxB; aceBA | Metabolism |
| 16 | iclR; gdhA; aceE | Lycopene |
| 17 | cra; edd; iclr | Metabolism |
| 18 | hnr; yliE | Lycopene |
| 19 | arcA | Metabolism |
| 20 | nudF | Isoprenol/Prenol |
| 21 | ptsHIcrr operon | Isoprenoids |
| 22 | pgi, gnd | Isoprene |
| 23 | gdhA, gpmA, gpmB, aceE, fdhF; talB; | Lycopene |
| | fdhF | |
| 24 | eno | Lycopene |
| 25 | pgk | Sugar metabolism |
| 26 | pgm | Sugar metabolism |
| 27 | gapA | Coenzyme Q10 |
| 28 | ptsG | Sugar metabolism |
| 29 | scpC | Poly hydroxy butyrate |
| 30 | asd | Amino acid metabolism |
| 31 | thrA | Metabolism |
| 32 | glnA | Bio-fuels |
| 33 | serA | Succinate |
| 34 | acc | Fatty acid metabolism |
| 35 | fabD | Fatty acid metabolism |
| 36 | ppK | Amino acid metabolism |
| 37 | glyA | Amino acid metabolism |
| 38 | rpiA | Sugar metabolism |
| 39 | pgl | Sugar metabolism |
| 40 | talA; talB | Sugar metabolism |
| 41 | tktB | Sugar metabolism |
| 42 | tktA | Sugar metabolism |
| 43 | rpe | Sugar metabolism |
| 44 | lldD | Lactate metabolism |
| 45 | tnaA | Amino acid metabolism |
| 46 | fnr; dpiA, crp, fur | Regulatory genes |
| 47 | gntK | Cofactor |

TABLE 2
Gene targets for overexpression.

| Sr. | | Process/Molecule |
|---|---|---|
| no | Gene name | targeted |
| 1 | adhE1, | Butanol |
| 2 | galP; glk | Metabolism |
| 3 | gld | Isoprene |
| 4 | ompF, ompE, ndk, cmk, fbaA, fbaB, | Lycopene |
| | ompF, ompC, adk, pfkA, pfkB, pgi, | |
| | pitA | |
| 5 | gapB, fbp, pckA | Riboflavin |
| 6 | ppc, pck | Succinate |
| 7 | pepcK | Succinate |
| 8 | yhfR | IPP, DMAPP, |
| | | Isopentenol |
| 9 | pck, pps, rpoS, appY, yjiD, ycgW, | Lycopene |
| | wrbA, atpE | |
| 10 | appy, crl, rpoS | Lycopene |
| 11 | yggV + lpxH + hisL + ppa + cdh | Isoprenol/prenol |
| 12 | Atp operon, nuo, cyoABCD, cydAB, | B-Carotene |
| | sucAB, talB, tktA, gltA, sdhABCD | |
| 13 | yajO, rib | Terpene |
| 14 | zwf, gnd | Riboflavin |
| 15 | glnAp1, 2, pps | Lycopene |
| 16 | glf, glk | Shikimic acid |
| 17 | erpA | Fe—S cluster |
| 18 | fldA; fpr | Fe—S Cluster |
| 19 | iscA | Isoprene |
| 20 | tpiA; ompN | Lycopene |
| 21 | osPT1 | Phosphate |
| | | metabolism |
| 22 | lpd | Central carbon |
| | | metabolism |
| 23 | icd | Central carbon |
| | | metabolism |
| 24 | acnA | Central carbon |
| | | metabolism |
| 25 | gltA | Central carbon |
| | | metabolism |
| 26 | pntAB | NADPH |
| 27 | yfjB | NADPH |
| 28 | ackA | Fe—S cluster |
| 29 | sufA sufBDCSE | Fe—S transporter |
| 30 | rocG | Cofactor |
| 31 | ppnK | Cofactor |

The disclosure further relates to a microorganism comprising chromosome of the said microorganism integrated with genes belonging to MEP pathway, optionally along with recombineering system including but not limiting to Lambda-RED recombinase system and superoperon.

In an embodiment, the microorganism of the instant invention causes enhanced production of metabolite including but not limiting to isoprene and isoprenoid.

In an embodiment, the microorganism is selected from a group comprising E. coli K-12, E. coli K-12 MG1655, E. coli BL21 (DE3)

Alternatively, the disclosure relates to a recombinant microorganism comprising component selected from a group comprising chromosome of the said microorganism integrated with genes belonging to MEP pathway, gene construct comprising genes belonging to MEP pathway, superoperon and Phage Lambda derived RED Recombination system, or any combination thereof, for the production of the metabolite in the recombinant microorganism.

The disclosure also relates to a gene construct comprising genes belonging to MEP pathway, superoperon and phage lambda derived RED recombination system, and a vector comprising the said gene construct for the production of the metabolite.

Additional embodiments and features of the present disclosure will be apparent to one of ordinary skill in art based upon description provided herein. Further, the description provides for Examples illustrating the embodiments provided herein. However, the provided examples should not be construed to limit the scope of the present disclosure.

### Examples

### Example 1: Illustration of Creating the ‘5′lacZ-P_{tac} ispS-CamR-3′lacZ’ Fusion Fragment by Fusion PCR

The ‘5′lacZ-P_{tac} ispS-CamR-3′ lacZ’ fusion fragment was made by fusing 4 different fragments viz. 5′lacZ, P_{tac}-ispS, CamR and 3′lacZ using a technique known as ‘Fusion PCR’/Overlap Extension PCR. The technique requires that the fragments to be fused have a region of overlap/homology of about 20 base pairs. This was achieved by amplifying individual fragment by primers which have an extended tail i.e. a sequence with homology to the adjacent fragment in the final fusion product.

Accordingly, the following reactions are set up for making the fragments. Each reaction utilized a commercially available DNA polymerase such as Phusion® or OneTaq®,

Step 1A—The region upstream to the 5′ region of lacZ gene was amplified from E. coli genomic DNA. The reverse primer used in the reaction had a P_{tac} tail so that a short region homologous to P_{tac} got incorporated in the product fragment

Step 1B—The region upstream to the 3′ region of lacZ gene was amplified from E. coli genomic DNA using the respective primers

Step 1C—The P_{tac} promoter fragment was amplified from the plasmid pGEX 2T using specific primers

Step 1D—The ispS gene fragment was amplified from the plasmid pMK. The forward primer in this case had a P_{tac} tail so that a short region homologous to P_{tac} got incorporated upstream to the ispS in the reaction product

Step 1E—The CamR (Chloramphenicol resistance) gene fragment was amplified from the plasmid pHCMC05. The forward primer in this case had an ispS tail while the reverse primer had a 3′ lacZ tail.

Step 2—Products fragments of Step 1C i.e. P_{tac} and 1D i.e. ispS were purified using commercially available kit and these fragments were then fused to each other in a fusion PCR reaction. The reaction utilized the P_{tac} forward primer and the ispS reverse primer. The individual fragments annealed to each other by the virtue of their tail/region of homology and were amplified as a complete fragment by the two primers during the PCR.

Step 3-Products fragments of Step 1A—5′ lacZ with P_{tac} tail,

Step 1B—3′ lacZ,

Step 1E—CamR with ispS and 3′ lacZ tail and

Step 2—P_{tac}-ispS were purified fused using the fusion PCR. Only two primers (each at the either extreme end) i.e. 5′ lacZ forward primer and 3′ lacZ reverse primer were required for the fusion PCR. As stated earlier the fragments annealed to each other by the virtue of their tail/region of homology and were amplified as a complete fragment by the two primers during the PCR.

### Example 2: Integration of Individual MEP Genes at Different Loci

The MEP genes were fused to the P_{tac} promoter and then fused with the appropriate locus flanks such as lacZ, pyrF, arsB, and the selection marker gene. Each fragment/flank of MEP genes had overlapping tails allowing the creating of a fusion fragment (illustrated in FIG. 4). The integration construct of MEP genes was transformed into the strains carrying k-RED genes after their induction. The transformants were verified using diagnostic colony PCR and confirmed by checking for growth on appropriate selection media. A 7 fold increase in isoprene titer was observed with the introduction of 3 genes comprising dxs, fni and ispS at different loci over the base strain of E. coli K-12 having k-RED (illustrated in table 4.

Successive MEP genes were introduced at different loci (illustrated in FIG. 4), wherein initial introduction of ispS and a single copy of fni showed isoprene levels which are significantly improved from those observed with the superoperon. Further, integration of a copy of dxs showed further enhancement (illustrated in table 4)

TABLE 4
Isoprene titers for strains with MEP genes from B. subtilis over expressed at different loci.

| | | | | Control |
|---|---|---|---|---|
| | | | Isoprene | Isoprene |
| Sr. No. | Parent Strain Genotype | Modification | titer (ppm) | titer (ppm) |
| 1 | E. coli K-12 AmyA::RED ampR | LacZ:: P_{tac} ispS camR | 0.78 | Nil |
| 2 | E. coli K-12 AmyA::RED ampRLacZ:: | pyrF::fni KanR | 2.83 | 0.78 |
| | P_{tac} ispS camR [symbol] | | | |
| 3 | E. coli K-12 AmyA::RED ampRLacZ:: | ArsB:: P_{tac} dxs PyrF | 5.30 | 2.83 |
| | P_{tac} ispS camR pyrF::fni KanR [symbol] | | | |

*control isoprene titer refers to isoprene produced by wild type strain.

TABLE 5
MEP genes overexpressed at separate loci.

| | | Isoprene | Control |
|---|---|---|---|
| | Modification: Genes overexpressed at | titer | Isoprene |
| Sr. No. | separate loci | (ppm) | titer (ppm) |
| 1 | P_{tac} ispS | 0.78 | Nil |
| 2 | P_{tac} ispS, P_{tac} fni | 2.83 | 0.78 |
| 3 | P_{tac} ispS, P_{tac} fni, P_{tac} dxs | 5.30 | 2.83 |
| 4 | P_{tac} ispS, P_{tac} fni P_{tac} P_{tac} dxs, P_{tac} dxr | 4.20 | 5.3 |

*control isoprene titer refers to isoprene produced by wildtype strain.

Further, individual introduction of the remaining MEP genes such as ispG, ispH and ispDF from B. subtilis did not result in any significant enhancements of isoprene. ispG and ispH code for iron-sulfur cluster proteins and its activity is dependent on E. coli genes fidA and fpr encoding flavodoxin and flavodoxin reductase believed to be responsible for electron transfer.

Introduction of these 2 genes did not show any enhancement in isoprene titer. Since ispG and ispH used in the integration is from B. subtilis, it is possible that the E. coli Fe—S systems is not able to transfer iron-sulfur clusters to ispG and ispH from B. subtilis. Over-expression of these 2 genes from E. coli is then attempted and as expected individually no enhancement in isoprene values is observed, whereas with the genes in combination, a respectable increase in isoprene is noted (illustrated in Table 6, serial no. 7).

TABLE 6
illustrates isoprene titers from strains with isoprene biosynthetic pathway genes ispDF,
ispG and ispH from E. coli integrated in the genome at different loci

| | | | | Control |
|---|---|---|---|---|
| Sr. | | | Isoprene | Isoprene |
| No. | Parent Strain Genotype | Modification | titer (ppm) | titer (ppm) |
| 1 | E. coli K-12 AmyA::RED ampR LacZ:: P_{tac} | rbsA-R:: P_{tac}-EcispDF- | 4.79 | 6.1 |
| | ispS camR pyrF::fni KanR ArsB:: P_{tac} dxs | GmR, | | |
| | PyrF | | | |
| 2 | E. coli K-12 AmyA::RED ampR LacZ:: P_{tac} | mtlA:: P_{tac}-EcispG- | 6.48 | 6.1 |
| | ispS camR pyrF::fni KanR ArsB:: P_{tac} dxs | TetR, | | |
| | PyrF | | | |
| 3 | E. coli K-12 AmyA::RED ampR LacZ:: P_{tac} | speC:: P_{tac}-EcispH- | 5.5 | 6.1 |
| | ispS camR pyrF::fni KanR ArsB:: P_{tac} dxs | SpcR, | | |
| | PyrF | | | |
| 4 | E. coli K-12 AmyA::RED ampR LacZ:: P_{tac} | speC:: P_{tac}-EcispH- | 5.2 | 6.1 |
| | ispS camR pyrF::fni KanR ArsB:: P_{tac} dxs | SpcR, | | |
| | PyrF mtlA:: P_{tac}-EcispG-TetR | | | |
| 5 | E. coli K-12 AmyA::RED ampR LacZ:: P_{tac} | speC:: P_{tac}-EcispH- | 4.1 | 6.1 |
| | ispS camR pyrF:fni KanR ArsB:: P_{tac} dxs | SpcR, | | |
| | PyrF rbsA-R:: P_{tac}-EcispDF-GmR | | | |
| 6 | E. coli K-12 AmyA::RED ampR LacZ:: P_{tac} | mtlA:: P_{tac}-EcispG- | 7.60 | 6.1 |
| | ispS camR pyrF::fni KanR ArsB:: P_{tac} dxs | TetR, | | |
| | PyrF rbsA-R:: P_{tac}-EcispDF-GmR | | | |
| 7 | E. coli K-12 AmyA::RED ampR LacZ:: P_{tac} | speC:: P_{tac}-EcispH- | 7.4 | 6.1 |
| | ispS camR pyrF::fni KanR ArsB:: P_{tac} dxs | SpcR, | | |
| | PyrF rbsA-R:: P_{tac}-EcispDF-GmR, mtlA:: | | | |
| | P_{tac}-EcispG-TetR | | | |

*control isoprene titer refers to isoprene produced by wild type strain.

TABLE 7
illustrates over expression of MEP genes at separate loci.

| | | Isoprene | Control |
|---|---|---|---|
| | | titer | Isoprene |
| Sr. No. | Modification: Genes overexpressed at separate loci | (ppm) | titer (ppm) |
| 1 | P_{tac} ispS, P_{tac} fni, P_{tac} dxs, P_{tac}-EcispDF | 4.79 | 6.1 |
| 2 | P_{tac} ispS, P_{tac} fni, P_{tac} dxs, P_{tac}-EcispG | 6.48 | 6.1 |
| 3 | P_{tac} ispS, P_{tac} fni, P_{tac} dxs, P_{tac}-EcispH | 5.5 | 6.1 |
| 4 | P_{tac} ispS, P_{tac} fni, P_{tac} dxs, P_{tac}-EcispG, P_{tac}-EcispH | 5.2 | 6.1 |
| 5 | P_{tac} ispS, P_{tac} fni, P_{tac} dxs, P_{tac}-EcispDF, P_{tac}-EcispH | 4.1 | 6.1 |
| 6 | P_{tac} ispS, P_{tac} fni, P_{tac} dxs, P_{tac}-EcispDF, P_{tac}-EcispG | 7.60 | 6.1 |
| 7 | P_{tac} ispS, P_{tac} fni, P_{tac} dxs, P_{tac}-EcispDF, P_{tac}-EcispG, P_{tac}- | 7.4 | 6.1 |
| | EcispH | | |

*control isoprene titer refers to isoprene produced by wild type strain.

It is observed that using different strains of E. coli (K12, MG1655, and BL21 (DE3) as hosts with ispS, dxs and fni expression constructs integrated at chromosomal loci varying levels of isoprene production were observed.

TABLE 8
illustrates comparison of isoprene titres with dxs, fni and ispS over expressed at
individual loci in different E. coli strains.

| Strain | E. coli K-12 | E. coli MG1655 | E. coli Bl21DE3 |
|---|---|---|---|
| Gene Modification | ampRLacZ:: P_{tac} | ampRLacZ:: P_{tac} ispS | ampRLacZ:: P_{tac} ispS |
| | ispS camR pyrF::fni | camR pyrF::fni KanR | camR pyrF::fni KanR |
| | KanR ArsB:: P_{tac} dxs | ArsB:: P_{tac} dxs PyrF | ArsB:: P_{tac} dxs PyrF |
| | PyrF | | |
| Isoprene titer | 7.8 | 10.3 | 1.77 |
| (ppm) | | | |

### Example 3: Introduction of Copies of MEP Genes at Chromosomal Loci Using Auxotrophy

In the first step, thyA locus was used to introduce ispS. The integration construct was made using ampicillin as the resistance marker. ThyA knockouts were selected on trimethoprim and ampicillin plates supplemented with thymine. This integration construct was made by using fusion PCR, ispS fused to the inducible P_{tac} along with about 1 kb regions of homology upstream and downstream of thyA gene. Correct integrations results in a thyA knockout replaced with the ispS gene. The thiamine auxotrophs were confirmed with diagnostic PCR and lack of growth on minimal M9 medium.

In the following step, the thyA gene knockout strain (thiamine auxotroph) with ispS gene was used as a host. The integration construct had fni fused to the inducible P_{tac} along with about 1 kb regions of homology upstream and downstream of metB gene and thyA gene. Electroporation and integration resulted in a metB knockout with fni and thyA, restoring the strain to thiamine prototrophy but the knockout of metB gene results in methionine auxotrophy and therefore mandates that selection media for the transformants include methionine.

The methionine auxotroph carrying ispS and fni was used as a subsequent host. The integration construct had nutritional selection gene metB fused with dxs under the P_{tac} promoter with flanking regions of the glnA gene. Knockout of the glnA gene created a glutamine auxotroph which had methionine requirement restored.

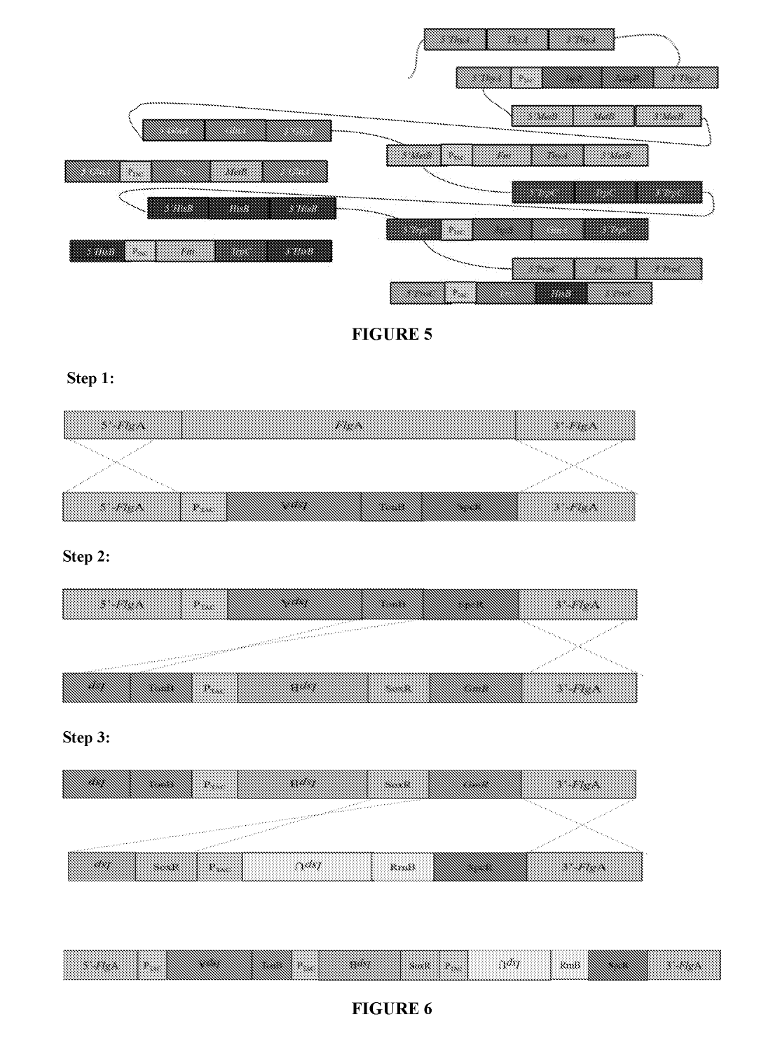

Thus, one copy each of the 3 main genes required for isoprene production, ispS, fin and dxs were introduced. Each successive integration in this manner created an auxotrophy which was complemented in the subsequent steps (illustrated in FIG. 5). The selection was carried out using media which lacks the amino acid/sugar of previous locus in minimal M9 medium but caters for the nutritional auxotrophy/requirement created by the knockout. This reduces the requirement for antibiotic selection genes and additional steps which are required for marker recycling.

The order of integration genes was ispS followed by fni and dxs; additional copy of ispS resulted in a decrease in isoprene titres which is augmented with the addition of another copy of fni (illustrated in table 9). Therefore, preferably, combination of at least one copy of MEP gene is essential for enhanced production of isoprene

TABLE 9
illustrates over expression of MEP genes at separate loci.

| | | | Control |
|---|---|---|---|
| | | Isoprene | Isoprene |
| | Modification: Genes | titer | titer |
| Sr. No. | overexpressed at auxotrophic loci | (ppm) | (ppm) |
| 1 | P_{tac} ispS, P_{tac} fni, P_{tac} dxs, | 7.8 | 2.83 |
| 2 | P_{tac} ispS, P_{tac} fni, P_{tac} dxs, P_{tac}-ispS | 5.40 | 7.8 |
| 3 | P_{tac} ispS, P_{tac} fni, P_{tac} dxs, P_{tac}-ispS, | 9.8 | 5.4 |
| | P_{tac} fni | | |
| 4. | P_{tac} ispS, P_{tac} fni, P_{tac} dxs, P_{tac}-ispS, | 8.3 | 9.8 |
| | P_{tac} fni, P_{tac} dxs | | |

### Example 4: Preparing Superoperon by Fusion PCR and Marker Switching

A superoperon is a strategy to stitch together multiple genes with their RBSs either under a single promoter or individual promoters. The strategy was expanded to either have the gene complex co-translated or as individual proteins. Stepwise preparation of ispA ispB and ispU superoperon by fusion PCR for use in the antisense based downregulation of these genes and marker switching is provided below—

Step 1: The first antisense (AS) gene in the superoperon was ispA. Primers were designed to insert the ispA gene in the reverse orientation so as to be complementary to the wild type gene. The 5′ and 3′ regions of the flgA locus, chosen as the site for integration of the superoperon, were amplified from the E. coli K-12 genome. The AS RNA fragment was also amplified from the genome and fused to a P_{tac} promoter. The primer used to amplify the AS ispA gene had a TonB terminator tail which would incorporate the TonB terminator downstream of the AS ispA sequence in the amplified product. The antibiotic marker used was spectinomycin and the primer for the same contained a tail bearing partial homology to the TonB sequence. The individually amplified products were fused and the fusion fragment was electroporated into the E. coli K-12 strain containing one copy of the ispS, dxs and fni genes at the flgA locus.

Step 2: The second gene in the superoperon was IspB. The AS IspB gene was amplified and fused to the P_{tac} promoter. The terminator attached as a primer tail in this case was SoxR. The antibiotic resistance used was gentamicin. The incomplete AS IspA-TonB fragment and the 3′ FlgA provided regions of homology for recombination. In this second integration event the spectinomycin resistance gene was replaced by the gentamicin resistance gene.

Step 3: The successive third round of transformation was with the fusion fragment containing the AS IspU gene with an RrnB terminator attached to it and the incomplete fragment of the previous IspB-SoxR. In this case the gentamicin resistance gene was replaced by the spectinomycin resistance gene for selection.

With each successive integration a section (preferably incomplete or upto 1 kb in size) of the previous gene and the 3′FlgA serve as the recombination regions and the previous marker (resistance) gene was switched, this allowed many successive genes to get replaced using only 2 resistance genes.

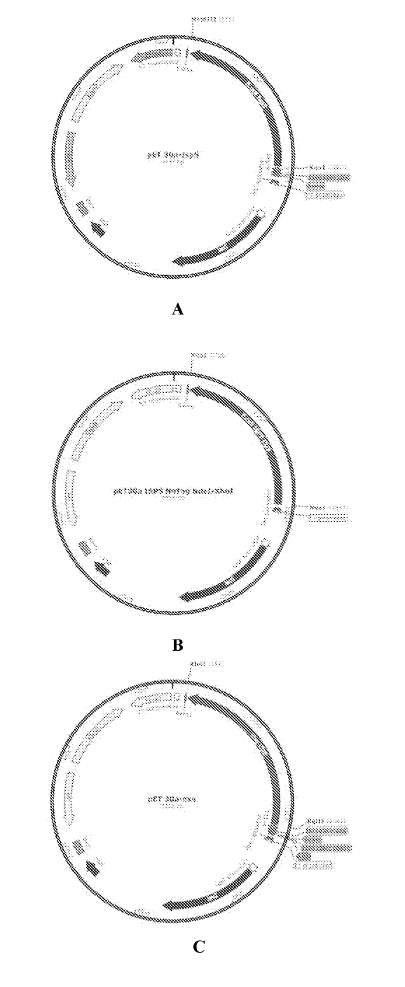

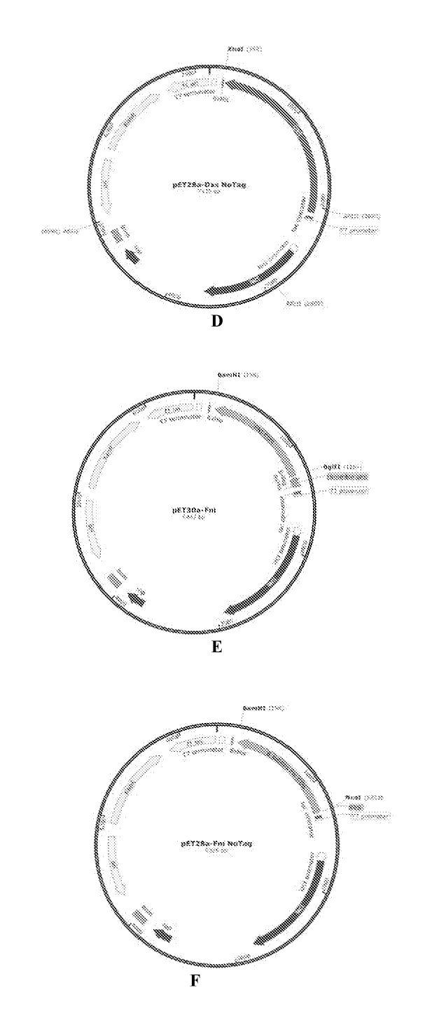

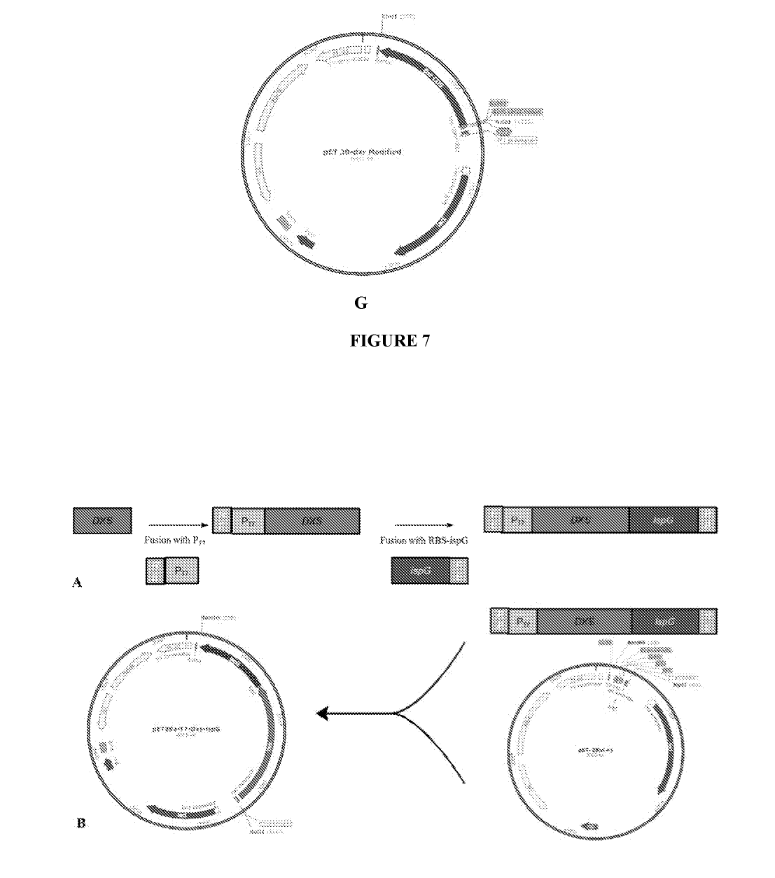

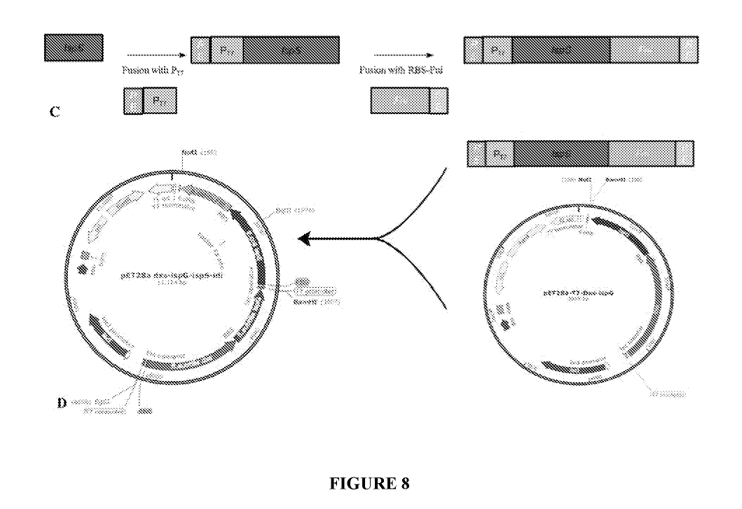

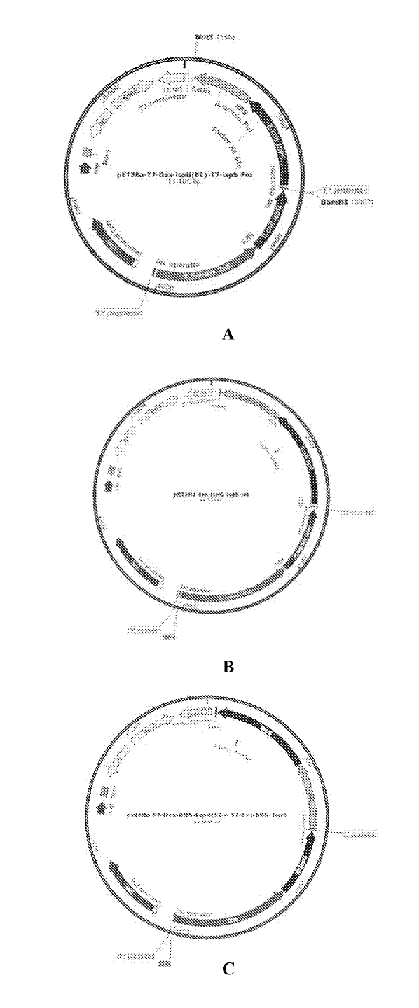

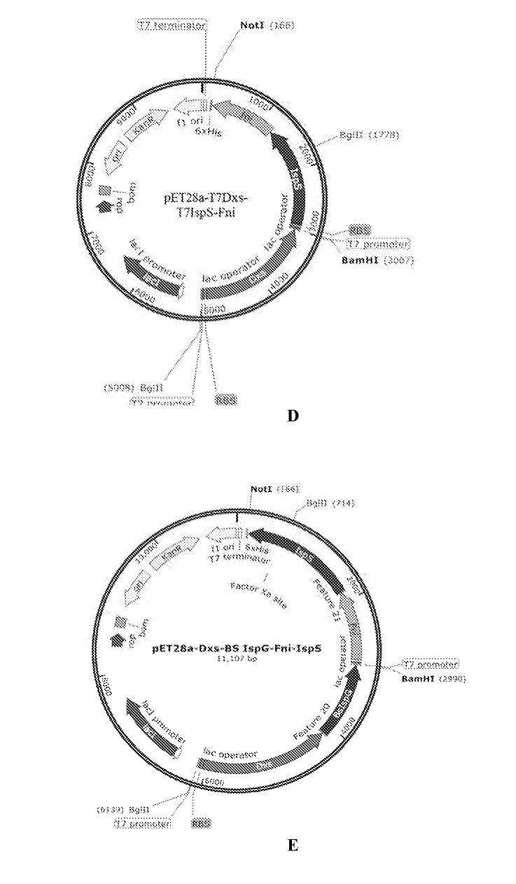

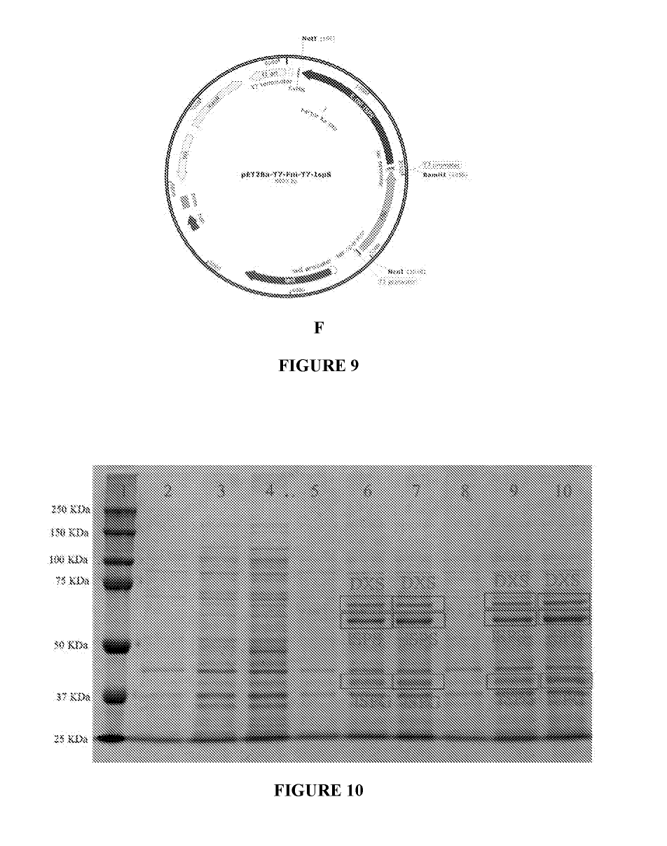

FIG. 6 illustrates the stepwise construction of superoperon.

### Example 5: T7-RNA Polymerase Carrying E. coli K 12 and MG1655 Strain for Production of Isoprene

T7 RNA polymerase from the bacteriophage T7 is highly specific to the strong T7 promoter, catalyzing the formation of RNA in the 5′-3′ direction. The T7 RNA polymerase begins transcription downstream of this T7 promoter so the gene of interest can be directly linked to the promoter. E. coli, BL21 (DE3) is the only strain which expresses the T7 RNA polymerase (under the PLACUV5 promoter along with the lacI gene) present at attB site between ybhC and xis loci. The T7 RNA polymerase gene was amplified from the BL21 (DE3) genome and integrated between the ybhB and ybhC loci on the E. coli K-12 and MG1655 genomes.

### Example 6: Cloning of MEP Genes for Expression

The MEP pathway genes are cloned into pET series of plasmids which use the T7 promoter for expression. The expression profiles and isoprene yields from these constructs is demonstrated in E. coli BL21 (DE3) strain, E. coli K-12 and E. coli K-12 MG1655.

The MEP genes are cloned into pET28a and pET30a with and without the His-Tag using standard cloning procedures. Plasmids with the MEP genes are illustrated in FIG. 5. Expression and induction profiles of these enzymes are studied. Purified proteins are used to generate antibodies for ispS, dxs and fni.

The expression plasmids with different combination of MEP genes are prepared by fusion PCR bypassing the stepwise cloning of each gene, the steps of preparation are illustrated below (FIG. 8 illustrates the cloning steps).
A. The promoter P_{T7} is amplified with a forward primer tail with a restriction site. The gene dxs is amplified with a forward primer with a tail with bases of the P_{T7}. ispG is amplified with a forward primer which has a tail with an RBS sequence and homology with dxs sequence, a reverse primer with a tail carrying a restriction site. In the first step P_{T7} and dxs are fused. In the next step the cleaned up fused P_{T7}-dxs fragment is then fused with ispG, leading to a fusion product with both restriction sites on both ends.
B. The fusion product is restriction digested and ligated with restriction digested pET28a. The ligation product is transformed and colonies are checked for the plasmid with colony PCR. The plasmid is confirmed with restriction digestion.
C. The promoter P_{T7} is amplified with a forward primer tail with a restriction site. The gene ispS is amplified with a forward primer with a tail with bases of the P_{T7}. ispG is amplified with a forward primer which has a tail with an RBS sequence and homology with dxs sequence and a reverse primer with a tail carrying a restriction site. In the first step P_{T7} and ispS are fused. In the next step the cleaned up fused P_{T7}-ispS fragment is then fused with fni. This gives a fusion product with both restriction sites on both ends.
D. This fusion product with ispS and fni is restriction digested and the plasmid carrying the ligated dxs and ispG in pET28a is restriction digested with the appropriate enzymes, then ligated and transformed. The final plasmid has all 4 genes expressing under P_{T7}.

The series of vectors carrying a combination of genes are generated. Each of these vectors is then transformed into the different T7 polymerase carrying hosts to check expression profiles and isoprene yields. The comparative fermentation profiles from the said vector construction are illustrated in table 10

TABLE 10
illustrates isoprene titers for different plasmid constructs having MEP genes

| | | | Isoprene | Isoprene | Isoprene |
|---|---|---|---|---|---|
| | | | (ppm) | (ppm) | (ppm) |
| | Host | | K-12 | MG1655 | BL21 (DE3) |
| Sr. No. | plasmid | Modification | Test | Test | Test |
| 1 | pET30a | ispS No His tag | 1.4 | 1.32 | |
| 2 | pET30a | ispS with His tag | 1.86 | 1.30 | |
| 3 | pET28a | T7-dxs-RBS-ispG (E. coli)- | 0.79 | 0.82 | 3.95 |
| | | T7-ispS-RBS-fni 7 | | | |
| 4 | pET28a | T7-dxs-RBS- | 11.6 | 9.85 | 8.26 |
| | | ispG (B. subtilis)-T7-ispS- | | | |
| | | RBS-fni | | | |
| 5 | pET28a | T7-dxs-T7-ispS-fni | 5.77 | 5.85 | 16.05 |
| 6 | pET28a | T7-dxs-T7-fni-ispS | 3.13 | 4.44 | 14.75 |
| 7 | pET28a | T7fni-T7-ispS | 2.95 | 11.76 | 13.4 |

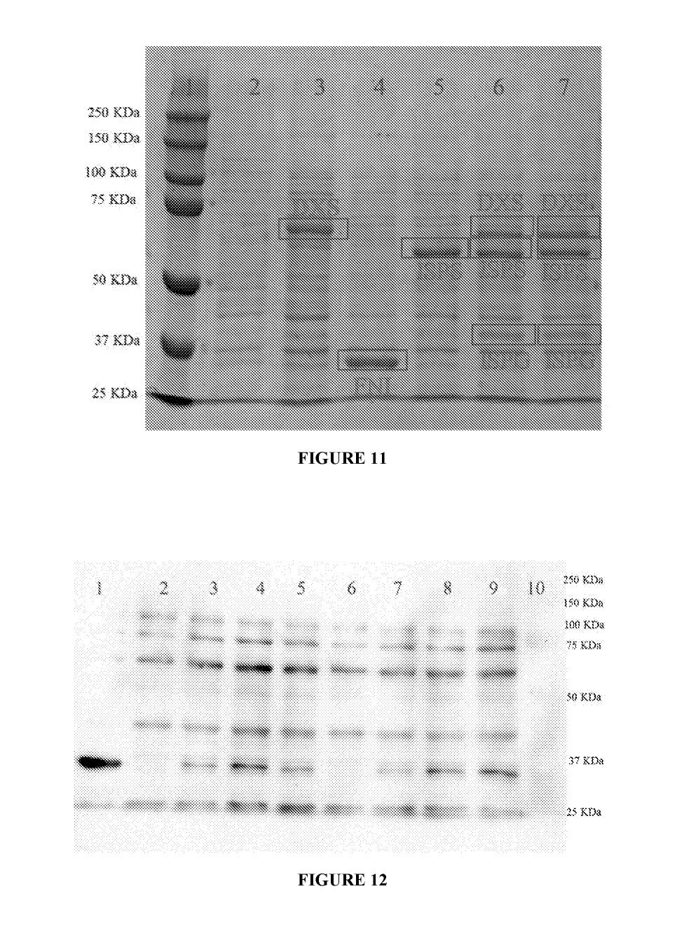

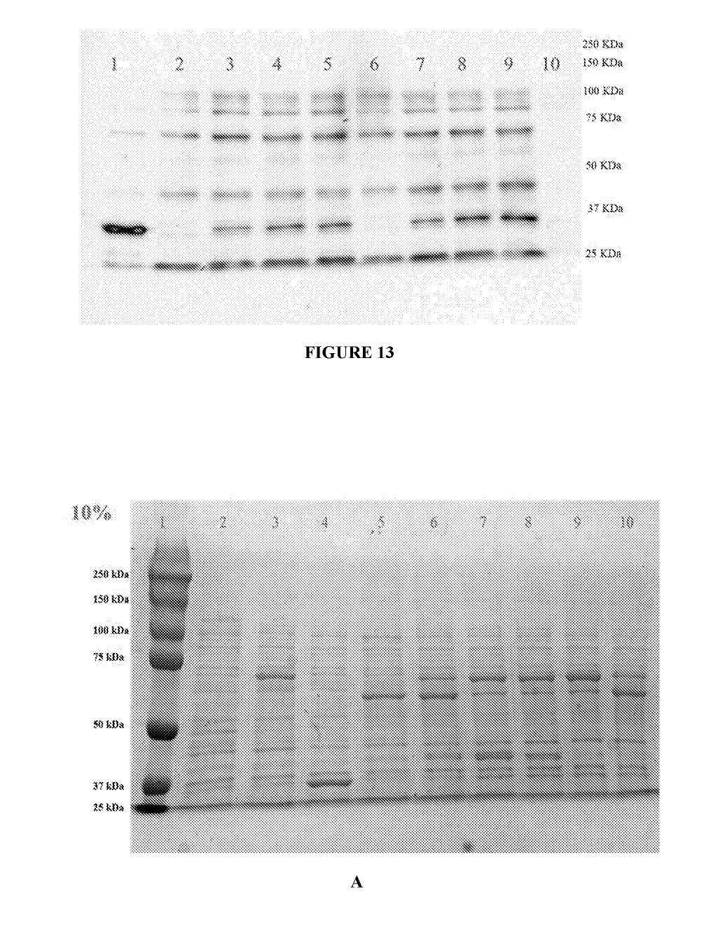

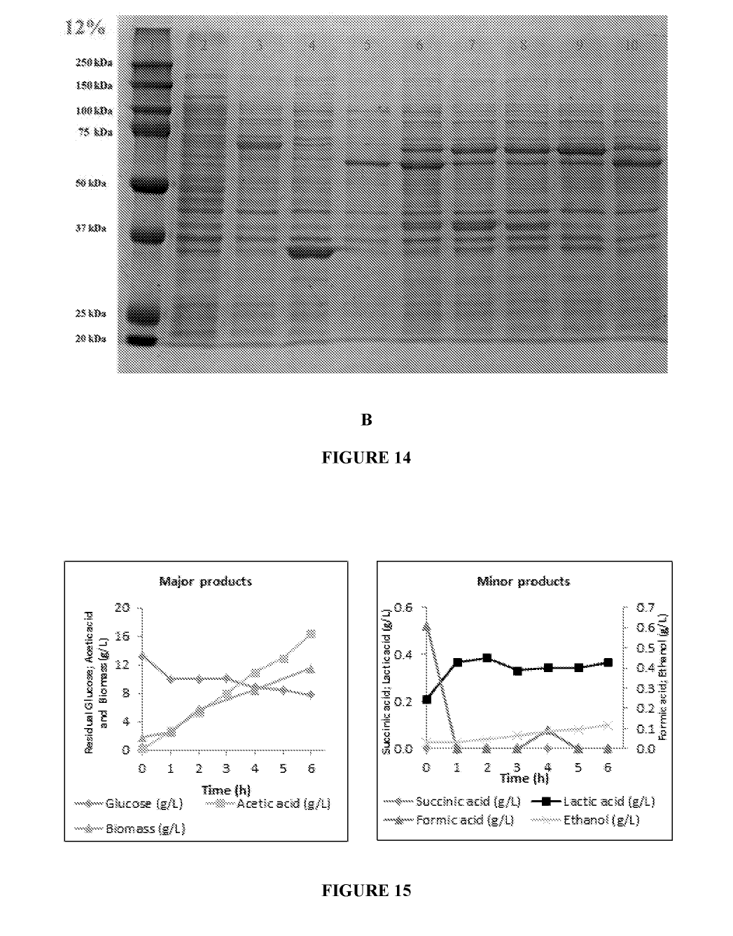

FIGS. 10 to 14 illustrate the protein expression of ispS, dxs, fni, ispG and dxr. From the expression profiles on the SDS-PAGE it is observed that FNI expression is at suboptimal levels and could not be detected in the gel. Western Blot analysis with FNI antibodies generated however showed the presence of the protein. FNI of 37.2 kDa which is detected in low amounts on the SDS gel is detected by Western blotting with anti-FNI antibodies. The low expression of FNI is due to the RBS and proximity of the gene to T7 promoter. Further gene constructs are made, wherein the gene order is switched such that the T7 Promoter is fused to fni, followed by RBS and ispS. Protein profiles indicated that FNI expression is better with a reduced level of ISPS as compared to the previous constructs.

### Example 7: Screening and Analysis of Modified Strains of E. coli for Isoprene Production

A. Production of Isoprene in Serum Vials Containing Modified E. coli Strains

Colonies of the modified strains of E. coli are inoculated in 100 ml Erlenmeyer shake flasks containing about 10 ml of sterilized (at about 121° C., about 10 minutes) M9 (Minimal) medium and kanamycin (about 50 μg/ml). E. coli cultures are grown overnight at about 37° C. with shaking at about 200 rpm. The OD600 of the overnight grown cultures is measured and about 10 to 15% (v/v) seed culture is inoculated in about 150 ml serum vials containing about 50 ml of M9 medium and the OD is normalized in each case. The composition of M9 medium in g/L: glucose, 10; Na₂HPO₄, 6.0; NaCl, 0.5; K₂HPO₄, 3.0; and (NH₄)₂Cl, 1.0; pH 6.8. Besides this, 2.0 ml of 1M MgSO₄ and 100 ul of 1.0 M CaCl₂.2H₂O per litre of medium was added separately after autoclaving. Modified Trace Metal Solution (1000×) 1.0 ml/L is added to the medium. The recipe for 1000× Modified Trace Metal Solution is: citric acid 40 g, MnSO4.H2O 30 g, NaCl 10 g, FeSO4.7H2O 1 g, CoCl2.6H2O 1 g, ZnSO4.7H2O 1 g, CuSO4.5H2O 100 mg, H3BO3 100 mg, NaMoO4.2H2O 100 mg. Each component is dissolved one at a time in milliQ water, pH is adjusted to about 3.0 with HCl/NaOH, and filter sterilized through a 0.221 filter. Kanamycin (about 50 μg/ml) and IPTG (0.1 mM) is added to the medium. The vials are sealed immediately with the rubber butyl bunks and aluminum crimps with the help of a crimper and incubated at about 37° to 40° C. with shaking at about 200 rpm for about 6 hours. After incubation, the vials are removed from the incubator and are heated at about 50° C. for about 10 minutes. Headspace (about 0.25 ml) of the sealed vials is withdrawn using gas tight syringe (about 2.5 ml, Hamilton) and injected on GC system (DB-1 column). The isoprene titers for different colonies of the modified strains are presented in Table 1-10.

B. Estimation of Isoprene, Residual Sugar and Metabolites

Isoprene calibration curve is plotted using standard isoprene at different concentrations (in ppm). The desired concentration of isoprene standards are prepared in about 150 ml bottles from the stock of isoprene prepared in water. For estimation of isoprene, headspace of the sealed vials (about 0.25 ml) is withdrawn using gas tight syringe (about 2.5 ml) and injected on GC system (DB-1 column).

The culture supernatant is analyzed for metabolites like acetic acid, lactic acid, succinic acid, ethanol and residual sugar by HPLC on organic acid column—HPX 87H (BioRad) with about 0.5 mM H₂SO₄ as the mobile phase using RI detector. Authentic standards of known concentrations of each metabolite are run to record the retention time and for plotting the curve.

The optical density is estimated using a UV spectrophotometer.

Intracellular metabolites or the intermediates of the MEP pathway are analyzed on LCMS using Kromasil C-18 column with a gradient mobile phase comprising of buffer A (0.1% v/v Formic acid in water) and an organic solvent B (0.1% v/v Formic acid in Acetonitrile). The gradient followed is mentioned in Table 11.

TABLE 11
Buffer gradient for LCMS

| Time (min.) | Composition of A (%) | Composition of B (%) |
|---|---|---|
| 0.0 | 100 | 0 |
| 1.8 | 100 | 0 |
| 3.10 | 60 | 40 |
| 4.90 | 60 | 40 |
| 5.40 | 10 | 90 |
| 9.50 | 10 | 90 |
| 10.0 | 100 | 0 |
| 15.0 | 100 | 0 |

Sample Preparation:

The modified strains grown in the fermenter is used for the extraction of intracellular metabolites. Sample is withdrawn at interval of one hour till the end of fermenter run. Five milliliter of the broth with OD 4 (at 600 nm) is used for intracellular metabolites analysis. Broth is withdrawn in the 50 ml falcon tubes (precooled at about −80° C.) and immediately centrifuged at about 5000 g at about −40° C. Supernatant is removed and the cell pellet suspended in cold water. The cell suspension is again centrifuged under same conditions to obtain cell pellet which is stored at about −80° C. for the intracellular metabolite extraction.

For extraction of metabolites, about 2 ml of extraction solution (Acetonitrile, methanol, water mixture [40:40:20] acidified with about 0.1M formic acid, cooled at about −80° C.) is added to the collected cell pellet. Cells are suspended in the extraction solution and kept at −20° C. for about 1 hour with intermittent shaking. The cells are then separated by centrifugation and supernatant collected. Second extraction is performed in same way using 1 ml of extraction solution. Both the supernatants are combined and stored at about −80° C. for analysis on LCMS.

Analysis protocol:

LCMS analysis was performed as described below.

HPLC Parameters:

Column: Kromasil C18, 150 mm×4.6 mm×3.5 μm

Buffer: (A) 0.1% Formic acid in water

Organic solvent: (B) 0.1% Formic acid in Acetonitrile

Mobile phase composition: Gradient as mentioned below

Column Temperature: 30° C.

Flow rate: 0.250 ml/min.

Injection volume: 10 μl

Run Time: 20 minutes

Mobile phase composition: Gradient (Below gradient programmed was followed)

| Time (min) | Composition of A (%) | Composition of B (%) |
|---|---|---|
| 0.0 | 100 | 0 |
| 1.8 | 100 | 0 |
| 3.10 | 60 | 40 |
| 4.90 | 60 | 40 |
| 5.40 | 10 | 90 |
| 9.50 | 10 | 90 |
| 10.0 | 100 | 0 |
| 15.0 | 100 | 0 |

MS parameters:

Ionization mode: Electrospray negative ionization

Capillary Voltage: 3500 V

Fragmentor Voltage: 150 V

Gas Temperature: 325° C.

Drying Gas flow: 10 L/min

Nebulizer pressure: 40 psig

Sheath gas temperature: 300° C.

Sheath gas flow: 11 L/min

LCMS data was then analyzed by mass scanning mode and retention time basis for the standard compounds from the MEP pathway. AUC for the detected metabolites compared and concentration expressed on the basis of ion abundance.

### Example 8: Fed Batch Fermentation

Fed-batch fermentations of modified E. coli strains are carried out in a 3.0 L bioreactor (Eppendorf, Bio Flow 115, Germany) containing about 1.0 L of M9 medium and Kanamycin (about 50 μg/ml). The fermenter is operated by feeding about 40 to 60% (w/v) solution of glucose and yeast extract. The pH is maintained between 6.8 and 7.2 using about 4.0 N NaOH/2N HCl, and the temperature is between 37° C. and 40° C. Foaming is controlled by adding silicon antifoam agent. Dissolved oxygen is maintained at about 35%-45% throughout the experiment by flushing air constantly into the medium at a flow rate of about 1.0-3.0 vvm through a sterile filter. Samples are collected at the regular intervals of about 0.5 h, processed for estimation of biomass, residual sugar and other metabolites. The isoprene present in the head space is analyzed by injecting 500 μl of the headspace gas into the GC. During the course of fermentation medium optimization, terrific broth was also used with some of the modified strains. Composition of terrific broth (TB) in g/L: glycerol 10; Yeast extract 24, Tryptone 12. These three components were added with other ingredients of M9 minimal medium to prepare TB medium.

Isoprene Production by Modified E. coli K-12 Strain

Fed batch fermentation using modified E. coli K-12 strain with three genes (ispS,fni and dxs) located at different loci is carried out. The genotype of the strain is E. coli K-12 AmyA::RED amp^{R} lacZ:: P_{tac} ispS camR pyrF:Ini KanR ArsB:: P_{tac} dxs PyrF. The initial fermentation medium contained about 1% (w/v) of glucose and yeast extract. The batch is fed with a total of 25.0 g/L (each) of glucose and yeast extract. The initial OD600 of batch is 5.0. The induction is carried out at 0 h using 0.1 mM IPTG. The DO is maintained at about 35%-45%. The isoprene concentrations with respect to time are presented in Table 12

TABLE 12
Isoprene production profile of modified E. coli K-12 strain

| | | Total isoprene | |
|---|---|---|---|
| Time | Isoprene conc in | flushed out* | IPTG (mM) |
| (h) | headspace (mgL⁻¹) | (mg) | addition |
| 0 | 0.00 | 0.00 | 0.1 |
| 0.5 | 0.15 | 2.54 | — |
| 1 | 0.38 | 8.46 | — |
| 2 | 0.49 | 13.39 | — |
| 2.5 | 0.54 | 15.57 | — |
| 3 | 0.51 | 15.59 | — |
| 3.5 | 0.47 | 14.61 | — |
| 4 | 0.37 | 12.48 | — |
| 4.5 | 0.31 | 10.10 | — |
| 5 | 0.37 | 10.30 | — |

*based on average isoprene concentration in head space between two time points along with delta change in isoprene in head space.

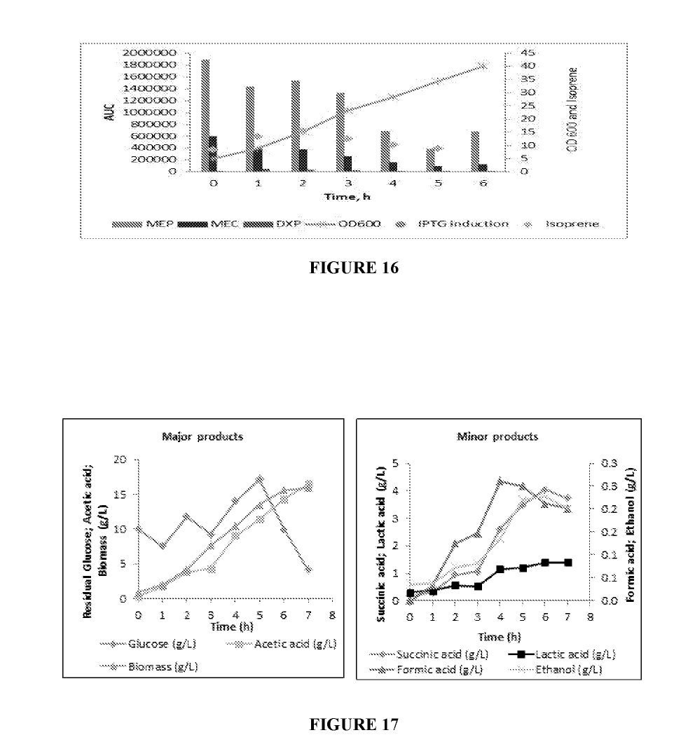

Total isoprene produced in about 5 hours is about 130.25 mg with a volumetric productivity of 21.7 mg/L/h, and a specific productivity of about 1.94 mg/g^{/}h⁻. The major and minor products are presented in FIG. 15 and the carbon balance is illustrated in Table 13. The intracellular metabolites are presented in FIG. 16.

TABLE 13
Carbon balance for fed batch fermentation
using modified E. coli K-12 strain

| | | Concentration | |
|---|---|---|---|
| Metabolites | | g/L | % carbon |
| Input | Consumed glucose | 29.28 | 48.88 |
| | YE carbon | 12.25 | 51.12 |
| Output | Succinic acid | 0.00 | 0.00 |
| | Lactic acid | 0.39 | 0.65 |
| | Formic acid | 0.00 | 0.00 |
| | Acetic acid | 16.37 | 27.33 |
| | Ethanol | 0.12 | 0.26 |
| | Biomass | 13.13 | 26.33 |
| | CO₂ | 11.18 | 12.72 |
| | Isoprene | 0.13 | 0.48 |

| | |
|---|---|
| Carbon accounted for | 67.77 |
| Carbon unaccounted for | 32.23 |

FIG. 16 illustrates that MEP is in excess initially and decreases over a period of time (about 3 h onwards).
DXP levels are found to be very low throughout the fermentation.
Accumulation of MEP could probably be a bottle neck leading to decreased overall productivity of isoprene.
Strategies to channel the accumulated MEP towards isoprene would help in mitigating the low productivity.

Isoprene Production by Modified E. coli K₁₂ Strain with Intermittent IPTG Induction

Fed batch fermentation using E. coli K₁₂ strain (three copies of ispS, fni and dxs each), was carried out for 8.0 h at 37° C. in M9 with initial glucose concentration of 10 g/L and 10 g/L of yeast extract. The DO was maintained at 35-40%. Culture was induced with 0.1 mM IPTG at 2.0 h and 4.5 h and 0.01 mM IPTG at 6.5 h. Culture was fed with 37.25 g of glucose and 30.0 g of yeast extract.

The isoprene concentration with respect to time is presented in Table 14.

TABLE 14
Isoprene production profile of modified E. coli K-12 strain

| | Total isoprene flushed | | |
|---|---|---|---|
| Time (h) | out* (mg) | OD (600 nm) | IPTG (mM) |
| 0 | 0.00 | 1.23 | — |
| 0.5 | 4.64 | — | — |
| 1 | 8.33 | 3.7 | — |
| 1.5 | 12.25 | — | — |
| 2 | 18.26 | 7.3 | 0.1 |
| 2.5 | 25.87 | — | — |
| 3 | 31.17 | 16.0 | — |
| 3.5 | 32.74 | — | — |
| 4 | 34.80 | 31.3 | — |
| 4.5 | 38.14 | — | 0.1 |
| 5 | 46.04 | 50.0 | — |
| 5.5 | 61.13 | — | — |
| 6.0 | 72.01 | 53.5 | — |
| 6.5 | 72.01 | — | 0.01 |
| 7.0 | 76.53 | 71.3 | — |
| 7.5 | 81.29 | — | — |
| 8.0 | 73.67 | 81.2 | — |

*based on average isoprene concentration in head space between two time points along with delta change in isoprene in head space

Total isoprene produced in 8 hours is about 688.8 mg with a volumetric productivity of 86.11 mg/L/h, and a specific productivity of about 3.26 mg/g^{/}h⁻. The major and minor products are presented in FIG. 17 and the carbon balance is illustrated in Table 15.

TABLE 15
Carbon balance for fed batch fermentation using
modified E. coli K-12 strain with intermittent IPTG induction

| | | Concentration | % |
|---|---|---|---|
| Metabolites | | in g/L | carbon |
| Carbon input | Consumed glucose | 43.38 | 68.18 |
| | YE carbon | — | 31.82 |
| Carbon output | Succinic acid | 0.01 | 0.02 |
| | Lactic acid | 0.00 | 0.00 |
| | Formic acid | 0.04 | 0.05 |
| | Acetic acid | 0.14 | 0.22 |
| | Propionic acid | 0.30 | 0.57 |
| | Ethanol | 0.01 | 0.02 |
| | Biomass | 23.17 | 43.76 |
| | CO₂ | 31.80 | 34.08 |
| | Isoprene | 0.69 | 2.39 |

| | |
|---|---|
| Carbon accounted for | 81.10 |
| Carbon unaccounted for | 8.90 |

Spiking of IPTG at different time points has led to the increase in isoprene titre compared to the previous batches with same strain where IPTG induction (0.1 mM) was done at the start of the batch.

Effect of Temperature on Isoprene Production by Modified E. coli MG1655 Strain:

The screening experiments conducted in the serum vials (150 ml) indicated that about 40° C. supports higher isoprene production, hence a fed batch fermentation using E. coli host strain MG1655 with three genes (ispS, fni and dxs) located at different loci is carried out at 40° C.

The genotype of the strain, E. coli MG1655 pTP223-Red Lac Z: P_{tac}-ispS Cam^{R} pyrF::P_{tac}-fni kan^{R} ArsB::P_{tac}-dxs PyrF. The initial fermentation medium contained about 1% (w/v) each of glucose and yeast extract. The batch is fed with 37.5 g of of glucose and 30 g of yeast extract. The isoprene concentrations with respect to time are presented in Table 16.

TABLE 16
Isoprene production profile of modified E. coli MG1655 strain

| | Isoprene | | |
|---|---|---|---|
| | concentration in | Total isoprene | |
| Time | headspace | flushed out* | IPTG (mM) |
| (h) | (mg/L) | (mg) | addition |
| 0 | 0 | 0 | 0.50 |
| 0.5 | 0.13 | 2.18 | — |
| 1.0 | 0.36 | 7.87 | — |
| 1.5 | 0.69 | 16.42 | — |
| 2 | 0.97 | 25.47 | — |
| 2.5 | 1.47 | 37.64 | — |
| 3 | 1.67 | 47.45 | 0.25 |
| 3.5 | 1.96 | 55.04 | — |
| 4 | 2.37 | 65.89 | 0.25 |
| 4.5 | 2.34 | 70.66 | — |
| 5 | 2.16 | 67.18 | — |

*based on average isoprene concentration in head space between two time points along with delta change in isoprene in head space.

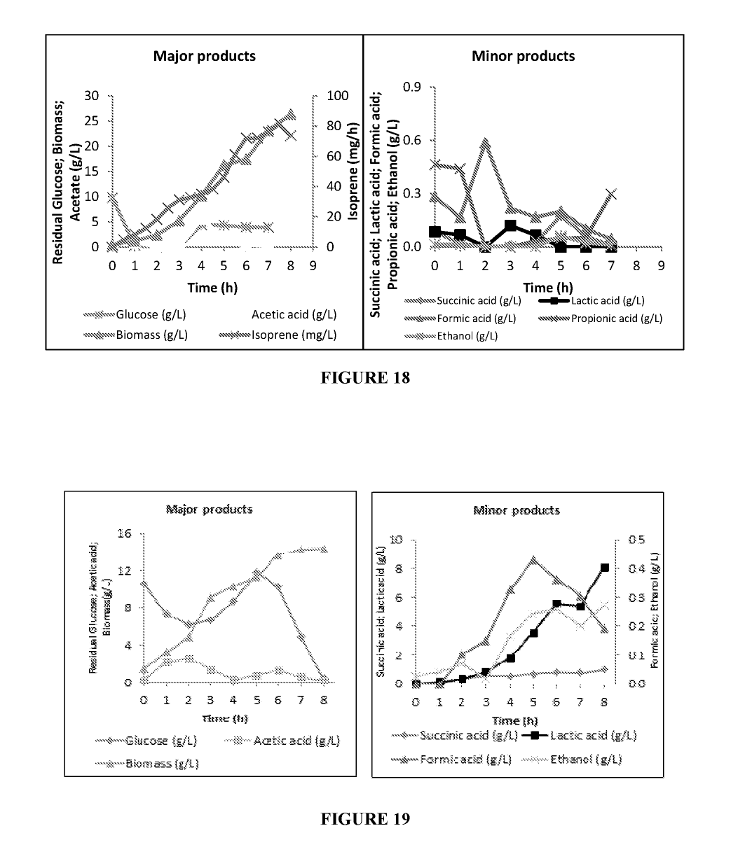

Total isoprene produced in about 5 hours is about 520.09 mg with a volumetric productivity of about 86.68 mg/L/h, and a specific productivity of about 5.5 mg/g/h. The major and minor products are presented in FIG. 18 and the carbon balance is illustrated in Table 17.

TABLE 17
Carbon balance for fed batch fermentation
using modified E. coli MG1655 strain

| | | Concentration | |
|---|---|---|---|
| Metabolites | | in g/L | % carbon |
| Input | Consumed glucose | 35.89 | 50.63 |
| | YE carbon | 14.00 | 49.37 |
| Output | Succinic acid | 4.05 | 5.82 |
| | Lactic acid | 1.40 | 1.97 |
| | Formic acid | 0.20 | 0.19 |
| | Acetic acid | 16.44 | 23.20 |
| | Ethanol | 0.20 | 0.37 |
| | Biomass | 15.89 | 26.93 |
| | CO₂ | 21.46 | 20.64 |
| | Isoprene | 0.52 | 1.62 |

| | |
|---|---|
| Carbon accounted for | 80.73 |
| Carbon unaccounted for | 19.27 |

Isoprene Production by Modified E. coli BL21 Strain:

Fed batch fermentation using modified strain of E. coli BL21 (DE3) having ispS gene dxs, fni and ispG genes (from Bacillus subtilis) on pET28a vector is carried out. The genotype of the strain is E. coli BL21 (DE3) pET28a T7 dxs RBS ispG (BS) T7 ispS RBS fni. The initial fermentation medium contained about 1% (w/v) of glucose and about 1% (w/v) yeast extract. The batch is fed with about 38.0 g/L each of glucose and yeast extract. The initial OD (at 600 nm) of batch is about 4.49. The DO is maintained at about 35% to 40%. The isoprene concentration with respect to time is presented in Table 18.

TABLE 18
Isoprene production profile of modified
E. coli BL21 (DE3) strain

| | Isoprene | | |
|---|---|---|---|
| | concentration in | Total isoprene | |
| Time | headspace | flushed out* | IPTG (mM) |
| (h) | (mg/L) | (mg) | addition |
| 0 | 0.00 | 0.00 | — |
| 0.5 | 0.90 | 15.23 | — |
| 1 | 1.19 | 31.83 | — |
| 1.5 | 1.33 | 38.10 | — |
| 2 | 1.76 | 47.31 | — |
| 2.5 | 1.70 | 51.78 | 0.2 |
| 3 | 2.28 | 60.77 | — |
| 3.5 | 4.28 | 102.29 | — |
| 4 | 3.87 | 121.43 | 0.1 |
| 4.5 | 3.84 | 115.57 | — |
| 5 | 4.10 | 119.52 | 0.1 |
| 5.5 | 3.24 | 108.30 | — |
| 6 | 3.06 | 94.09 | — |
| 6.5 | 2.32 | 79.26 | 0.2 |
| 7 | 1.97 | 63.73 | — |
| 7.5 | 1.78 | 55.90 | — |

*based on average isoprene concentration in head space between two time points along with delta change in isoprene in head space.

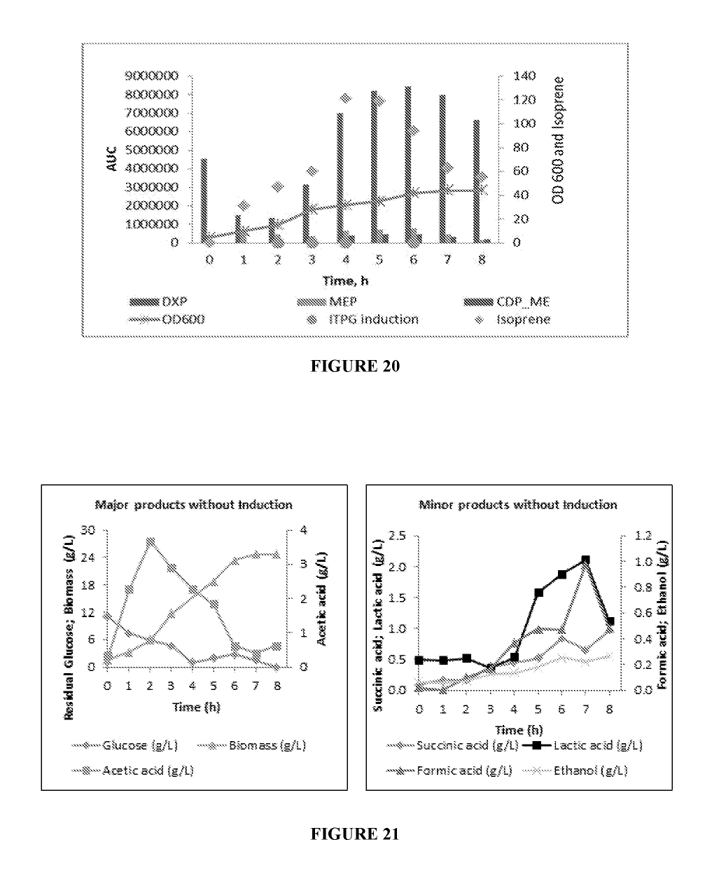

Total isoprene produced in about 7.5 hours is about 1105.11 mg at a volumetric productivity of about 147.35 mg/L^{/}h and a specific productivity of about 10.30 mg/g/h. The major and minor products are presented in FIG. 19 and the carbon balance is illustrated in Table 19. The intracellular metabolites are presented in FIG. 20.

TABLE 19
Carbon balance for fed batch fermentation
using modified E. coli BL21 strain

| | | Concentration | |
|---|---|---|---|
| Metabolites | | in g/L | % carbon |
| Input | Consumed glucose | 47.79 | 53.48 |
| | YE carbon | 16.63 | 46.52 |
| Output | Succinic acid | 0.98 | 1.11 |
| | Lactic acid | 8.07 | 9.03 |
| | Formic acid | 0.43 | 0.31 |
| | Acetic acid | 2.56 | 2.87 |
| | Ethanol | 0.27 | 0.40 |
| | Biomass | 14.37 | 19.32 |
| | CO₂ | 43.80 | 33.42 |
| | Isoprene | 1.09 | 2.68 |

| | |
|---|---|
| Carbon accounted for | 69.14 |
| Carbon unaccounted for | 30.86 |

Isoprene production by modified E. coli BL21 strain (without induction): The fermentation medium contained about 1% (w/v) of glucose and about 1% (w/v) yeast extract. The batch is fed with about 60.0 g/L of glucose and about 47.5 g/L yeast extract. The initial OD (at 600 nm) of batch is about 5.0. The DO is maintained at about 35% to 40%. The isoprene concentration with respect to time are illustrate in Table 20.

TABLE 20
Isoprene production profile of modified E. coli BL21 (DE3) strain
(without induction)

| | Isoprene | |
|---|---|---|
| | concentration in | Total isoprene |
| Time | headspace | flushed out* |
| (h) | (mg/L) | (mg) |
| 0 | 0.00 | 0.00 |
| 0.5 | 0.80 | 13.56 |
| 1 | 1.08 | 28.65 |
| 1.5 | 0.91 | 29.39 |
| 2.0 | 1.01 | 29.03 |
| 2.5 | 1.40 | 36.96 |
| 3.0 | 1.93 | 51.02 |
| 3.5 | 2.94 | 75.03 |
| 4.0 | 3.13 | 91.36 |
| 4.5 | 3.40 | 98.56 |
| 5.0 | 4.18 | 115.29 |
| 5.5 | 5.09 | 140.91 |
| 6.0 | 5.98 | 167.88 |
| 6.5 | 5.52 | 171.54 |
| 7.0 | 5.78 | 169.99 |
| 7.5 | 5.33 | 165.83 |
| 8.0 | 2.91 | 118.87 |
| 8.5 | 2.15 | 74.51 |
| 9.0 | 0.95 | 44.21 |

*based on average isoprene concentration in head space between two time points along with delta change in isoprene in head space.

Total isoprene produced in about 9 hours is about 1622.59 mg at a volumetric productivity of 180.29 mgL⁻¹ h⁻¹ and a specific productivity of about 7.30 mg/g/h. The major and minor products are presented in FIG. 21 and the carbon balance is illustrated in Table 21.

TABLE 21
Carbon balance for fed batch fermentation using
modified E. coli BL21 strain (without induction)

| | | Concentration | |
|---|---|---|---|
| Metabolites | | in g/L | % carbon |
| Input | Consumed glucose | 70.98 | 58.52 |
| | YE carbon | 20.13 | 41.48 |
| Output | Succinic acid | 0.97 | 0.81 |
| | Lactic acid | 2.11 | 1.74 |
| | Formic acid | 0.98 | 0.53 |
| | Acetic acid | 3.68 | 3.03 |
| | Ethanol | 0.26 | 0.28 |
| | Biomass | 24.70 | 24.47 |
| | CO₂ | 50.29 | 28.27 |
| | Isoprene | 1.62 | 2.95 |

| | |
|---|---|
| Carbon accounted for | 62.08 |
| Carbon unaccounted for | 37.92 |

The data indicates that BL21 (DE 3) strain has leaky expression yielding higher isoprene titres without IPTG induction. It is also observed that the biomass production is higher (about 24.7 g/L) under non-induced conditions as compared to induced condition (about 14.3 g/L). This indicates that the inducer (IPTG) retards the growth of the cells. Acetic acid production increased up to 2.0 h in both the cases and thereafter declined even though glucose is being continuously supplied. Lactic acid production is higher under induced condition (8.07 g/L) as compared to non-induced condition (2.1 g/L).

Therefore, indicating that higher the copy numbers of MEP genes on the chromosomes better is the isoprene production in the modified construct. Here, the copy number of the MEG genes on the chromosomes is increased using auxotrophy.

Isoprene Production by Modified E. coli BL21 Strain Using Terrific Broth with Glycerol as Feed

Fed batch fermentation using a E. coli BL21 (DE3) strain (IspS, DxS, Fni and IspG on pET 28a vector), was carried out for 9.0 h at 37° C. in TB medium with initial glycerol concentration of 10 g/L, 24 g/L of yeast extract and 12 g/L Tryptone. The DO was maintained at 35-40%. The batch was run with higher aeration rate of 3 vvm. Kanamycin was added to medium initially at the concentration of 50 ug/ml.

The batch was fed with 121 g of glycerol, 40 g of yeast extract and 20 g of tryptone. The initial OD600 of the batch was 3.80. The DO is maintained at about 35% to 40%. The pH was maintained at 6.8 from the beginning of the batch. The isoprene concentration with respect to time is illustrated in Table 22.

TABLE 22
Isoprene production profile of modified E. coli
BL21 (DE3) strain using terrific broth as culture medium

| | Total isoprene flushed | |
|---|---|---|
| Time (h) | out* (mg) | OD (600 nm) |
| 0 | 0.00 | 3.8 |
| 0.5 | 0.00 | — |
| 1 | 5.34 | 5.5 |
| 1.5 | 16.78 | — |
| 2 | 25.01 | 9.0 |
| 2.5 | 34.17 | — |
| 3 | 46.76 | 24.0 |
| 3.5 | 90.80 | — |
| 4 | 148.19 | 40.0 |
| 4.5 | 192.17 | — |
| 5 | 219.66 | 46.5 |
| 5.5 | 239.20 | — |
| 6.0 | 267.36 | 65.0 |
| 6.5 | 303.34 | — |
| 7.0 | 330.92 | 77.0 |
| 7.5 | 413.95 | — |
| 8.0 | 427.03 | 91.8 |
| 8.5 | 330.90 | — |
| 9.0 | 226.38 | 100.2 |

*based on average isoprene concentration in head space between two time points along with delta change in isoprene in head space

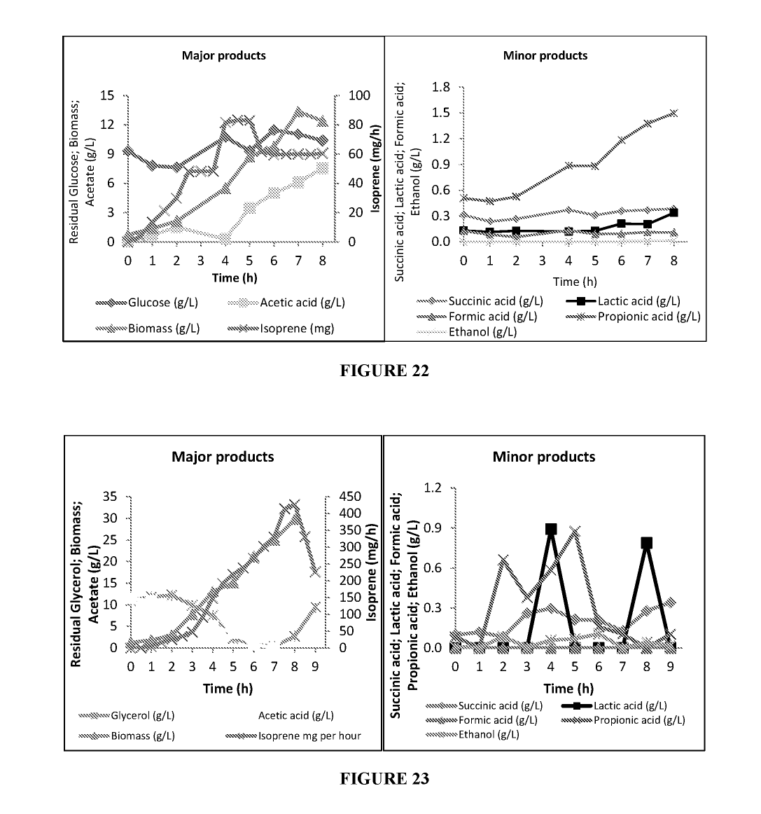

Total isoprene produced in about 9 hours is about 3318 mg/L at a volumetric productivity of 368.66 mgL⁻¹ h⁻¹ and a specific productivity of about 11.32 mg/g/h. The major and minor products are presented in FIG. 22 and the carbon balance is illustrated in Table 23

TABLE 23
Carbon balance for fed batch fermentation using modified
E. coil BL21 strain with terrific broth as culture medium

| | | Concentration | % |
|---|---|---|---|
| Metabolites | | in g/L | carbon |
| Carbon input | Consumed glycerol | 54.34 | 98.48 |
| | YE carbon | — | 1.52 |
| Carbon output | Succinic acid | 0.34 | 0.03 |
| | Lactic acid | 0.00 | 0.00 |
| | Formic acid | 0.00 | 0.00 |
| | Acetic acid | 12.33 | 1.89 |
| | Propionic acid | 0.10 | 0.23 |
| | Ethanol | 0.02 | 0.00 |
| | Biomass | 32.57 | 49.33 |
| | CO₂ | 42.00 | 29.56 |
| | Isoprene | 3.32 | 7.23 |

| | |
|---|---|
| Carbon accounted for | 88.26 |
| Carbon unaccounted for | 11.74 |

The titre and volumetric productivity achieved is the highest with this strain, because glycerol metabolism allows production of Glyceraldehyde 3 phosphate (G3P), with less metabolic burden as compared to glucose. G3P biosynthesis is the limiting step in isoprene production. Hence, it was seen that glycerol is a better carbon source for the production of isoprene via the MEP pathway.

Comparison of Recovery of Carbon Using the Modified Strains with Glucose as Carbon Source

The isoprene titres (mg/L) as obtained in 1.0 L bioreactor for different modified strains of E. coli i.e. K-12 (having one copy each of ispS, fni and dxs integrated in the chromosome), MG1655 (having one copy each of ispS, fni and dxs integrated in the chromosome) and BL21 (DE3) (having ispS, fni, dxs and ispG on plasmid) (without IPTG induction) about 130, about 520 and about 1622, respectively. The amount of carbon recovered in the form of isoprene is about 0.48, about 1.62 and about 2.95% (in carbon moles), respectively, illustrated in table 24.

TABLE 24
Comparison of recovery of carbon using the modified strains

| | Recovery of carbon in mole-% | | |
|---|---|---|---|
| | | | BL21(DE3) |
| | | | Without |
| Metabolite | K-12 | MG 1655 | Induction |
| Lactic acid | 0.65 | 1.97 | 1.74 |
| Acetic acid | 27.33 | 23.20 | 3.03 |
| Biomass | 26.33 | 26.93 | 24.47 |
| CO₂ | 12.72 | 20.64 | 28.27 |
| Isoprene | 0.48 | 1.62 | 2.95 |

The results illustrated in Table 23 indicates that having multiple copies of the MEP pathway genes dxs, ispG, fni and ispS on plasmid in the modified E. coli BL21 (DE3) strain has improved the flux of carbon towards the production of isoprene. The glucose consumption rate for the modified E. coli strains K-12, MG 1655 and BL21 is about 4.88, about 5.12 and about 8.8 g/L/h, respectively, illustrating positive correlation between glucose consumption with isoprene production. It is also observed that as the carbon availability towards acetate production decreases, the amount of carbon flux towards isoprene increases.

The amount of carbon fluxed towards succinic acid, formic acid and ethanol is negligible compared to acetic acid and lactic acid in all the three strains. Thus, the major distribution of carbon is towards biomass, carbon dioxide, acetic acid and lactic acid, thereby resulting in relatively lower carbon availability for isoprene production, indicating that the carbon flux towards isoprene production is essential

Fed Batch Fermentation for Isoprene Production by MG1655 Strain with Three Copies of ispS, and Two Copies Dxs and Fni Genes

Fed batch fermentation using MG1655 with three copies of ispS and two copies of dxs, and fni at trypA (tryptophan auxotrophy) locus is carried out at 40° C. The batch is fed with glucose (25.0 g/L) and yeast extract (10 g/L). The initial fermentation medium contained about 1% of glucose and about 1% yeast extract. The initial OD (at 600 nm) of the culture is 2.20. The batch is run with 0 h IPTG induction (0.5 mM). The DO is maintained at 35% to 45%.

The isoprene concentration with respect to time are illustrate in Table 25.

TABLE 25
Isoprene production profile of using modified E. coil MG1655 strain
with three copies of ispS and two copies each of dxs and fni genes

| | Isoprene | |
|---|---|---|
| | concentration in | Total isoprene |
| | headspace | flushed out* |
| Time (h) | (mg/L) | (mg) |
| 0 | 0.00 | 0.00 |
| 0.5 | 0.22 | 3.67 |
| 1 | 0.63 | 13.48 |
| 1.5 | 0.78 | 21.34 |
| 2 | 1.17 | 30.03 |
| 3.5 | 2.00 | 144.64 |
| 4 | 3.26 | 81.51 |
| 5 | 2.34 | 166.16 |
| 5.5 | 1.84 | 61.68 |
| 6 | 1.97 | 57.50 |
| 6.5 | 2.02 | 59.96 |
| 7 | 1.84 | 57.49 |
| 7.5 | 2.11 | 59.78 |
| 8 | 1.95 | 60.55 |

*based on average isoprene concentration in head space between two time points along with delta change in isoprene in head space

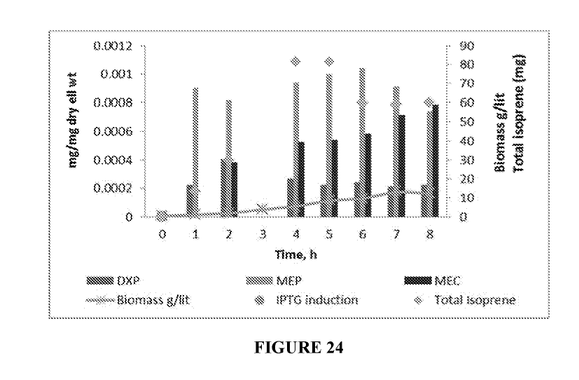

Total isoprene produced in about 8.0 hours is about 817.0 mg at a volumetric productivity of about 102.1 mg/L/h and a specific productivity of about 8.27 mg/g/h. The major and minor products are presented in FIG. 23 and the carbon balance is illustrated in Table 26. The intracellular metabolites are presented in FIG. 24.

TABLE 26
Carbon balance for fed batch fermentation using
modified E. coli MG1655 strain with three copies
of ispS and two copies each of dxs and fni genes

| | Concentration | % |
|---|---|---|
| Metabolites | in g/L | carbon |
| Consumed glucose | 23.92 | 47.68 |
| YE carbon | 30 | 52.32 |
| Succinic acid | 0.39 | 0.78 |
| Lactic acid | 0.34 | 0.67 |
| Formic acid | 0.11 | 0.14 |
| Acetic acid | 7.56 | 15.06 |
| Propionic acid | 1.50 | 3.63 |
| Ethanol | 0.01 | 0.03 |
| Biomass | 12.38 | 29.65 |
| CO₂ | 6.95 | 9.44 |
| Isoprene | 0.82 | 3.59 |

| | |
|---|---|
| Carbon accounted for | 92.17 |
| Carbon unaccounted for | 7.83 |

An increased carbon flux (3.59%) towards isoprene is obtained using the modified E. coli strain with three copies of ispS and two copies of ctcs, and fni. The MEP and MEC concentration increased over time with constant levels of DXP, indicating the need of these to be channelled further down to DMAPP. An ispG gene over expression that converts MEC to HMBPP would possibly be helpful in funneling the carbon flux towards isoprene.

We claim:

1. A method for enhanced production of metabolite selected from a group comprising isoprene and isoprenoid, or a combination thereof, said method comprising steps of: transforming host cell with gene construct comprising 2-C-methyl-D-erythritol 4-phosphate (MEP) genes; and integrating the MEP genes into chromosome of the host cell, thereby enhancing production of the metabolite selected from the group comprising isoprene and isoprenoid.

2. The method as claimed in claim 1, wherein the host cell is selected from a group comprising bacteria and fungi.

3. The method as claimed in claim 2, wherein the host cell is selected from a group comprising E. coli K-12, E. coli K-12 MG1655 and E. coli BL21.

4. The method as claimed in claim 1, wherein the MEP genes is selected from a group comprising dxs, fni, idi, dxr, ispG, ispS, ispD, and ispF, or any combination thereof.

5. The method as claimed in claim 4, the MEP genes are fused by fusion PCR prior to transformation into the host cell, wherein the MEP genes are fused in a combination selected from a group comprising—
isps and fni;
isps, fni and dxs;
isps, fni, dxs, dxr and
isps, fni, dxs, ispD;

6. The method as claimed in claim 5, wherein the fused MEP genes comprises auxotrophic markers selected from a group comprising thyA, metB, glnA, trpA, leuA, tyrA, lysA and proC, or a combination thereof.

7. The method as claimed in claim 1, wherein the host cell comprises lambda RED recombination system and superoperon.

8. The method as claimed in claim 7, wherein the superoperon comprises MEP genes separated by internal ribosome entry site and accessory genes selected from a group comprising Fe—S cluster interacting redox polypeptides and co-factor balancing genes, or a combination thereof.

9. The method as claimed in claim 1, wherein the integration of the MEP genes with the chromosome of the host cell is by lambda RED recombination system in the host cell.

10. The method as claimed in claim 1, wherein the volumetric productivity of the isoprene is ranging from about 86.0 mg L⁻¹ h⁻¹ to 102 mg L⁻¹ h⁻¹ and the specific productivity of the isoprene is ranging from about 3.26 mg g⁻¹ h⁻¹ to 8.27 mg g⁻¹ h⁻¹.

11. A gene construct comprising MEP genes and auxotrophic marker or antibiotic resistance marker or a combination thereof.

12. The gene construct as claimed in claim 11, wherein the MEP genes are selected from a group comprising dxs, fni, idi, dxr, ispG, ispS, ispD, and ispF, or any combination thereof; and the auxotrophic marker is selected from a group comprising thyA, metB, glnA, trpA, leuA, tyrA, lysA and proC, or a combination thereof.

13. The gene construct as claimed in claim 11, wherein the gene construct comprises MEP genes in a combination selected from a group comprising—
isps and fni;
isps, fni and dxs;
isps, fni, dxs, dxr and
isps, fni, dxs, ispD;
along with the auxotrophic marker

14. A host cell comprising chromosome integrated with MEP genes, lambda RED recombination system and superoperon.

15. The host cell as claimed in claim 14, wherein the host cell is selected from a group comprising bacteria and fungi.

16. The host cell as claimed in claim 14, wherein the host cell is selected from a group comprising E. coli K-12, E. coli K-12 MG1655 and E. coli BL21.

17. The host cell as claimed in claim 14, wherein the superoperon comprises MEP genes separated by internal ribosome entry site and accessory genes selected from a group comprising Fe—S cluster interacting redox polypeptides and co-factor balancing genes, or a combination thereof.

18. The host cell as claimed in claim 14, wherein the cell produces enhanced isoprene with volumetric productivity of the isoprene ranging from about 86.1 mg L⁻¹ h⁻¹ to 102.1 mg L⁻¹ h⁻¹ and the specific productivity of the isoprene ranging from about 3.26 mg g⁻¹ h⁻ to 8.27 mg g⁻¹ h⁻.

19. A vector comprising the gene construct defined in claim 11.
